SEQUENCE LISTING (11 sequences)

SEQ ID NO: 1 - DNA, 2028 bp, Homo sapiens
  misc_feature: Human Ig germline H-chain G-E-A region B gamma-4 constant region, 3′ end
agctttctggggcaggccgggcctgactttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctcccggcaccacaggctggatgcccctaccccaggccctgcgcatacagggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgagggaggcagagcgggtcccactgtccccacactgg

SEQ ID NO: 2 - DNA, 61 bp, Homo sapiens
  misc_feature: IgG4 Hinge Region Nucleic Acid
tctgcagagtccaaatatggtcccccatgcccatcatgcccaggtaagccaacccaggcct

SEQ ID NO: 3 - protein, 12 aa, Homo sapiens
  misc_feature: IgG4 Hinge Region Amino Acid
ESKYGPPCPSCP

SEQ ID NO: 4 - DNA, 33 bp, artificial sequence
  misc_feature: S241P Oligo Nucleic Acid
ggtcccccatgtcctccctgcccaggtaagcca

SEQ ID NO: 5 - protein, 11 aa, artificial sequence
  misc_feature: S241P Oligo Amino Acid
GPPCPPCPGKP

SEQ ID NO: 6 - DNA, 65 bp, Homo sapiens
  misc_feature: IgG4 Hinge Region Nucleic Acid
cttctctctgcagagtccaaatatggtcccccatgcccatcatgcccaggtccgccaacccaggc

SEQ ID NO: 7 - protein, 12 aa, Homo sapiens
  misc_feature: IgG4 Hinge Region Amino Acid
ESKYGPPCPSCP

SEQ ID NO: 8 - DNA, 65 bp, Homo sapiens
  misc_feature: IgG2 Hinge Region Nucleic Acid
cttctctctgcagagcgcaaatgttgtgtcgagtgcccaccgtgcccaggtccgccaacccaggc

SEQ ID NO: 9 - protein, 12 aa, Homo sapiens
  misc_feature: IgG2 Hinge Region Amino Acid
ERKCCVECPPCP

SEQ ID NO: 10 - DNA, 33 bp, artificial sequence
  misc_feature: Oligo 2014 Nucleic Acid
gaggagcagttccagtctacttaccgagtggtc

SEQ ID NO: 11 - protein, 11 aa, artificial sequence
  misc_feature: Oligo 2014 Amino Acid
EEQFQSTYRVV

(19) United States
(12) Patent Application Publication
(10) Pub. No.: US 2005/0097625 A1
(43) Pub. Date: May 5, 2005

(54) Modified antibodies stably produced in milk and methods of producing same

(76) Inventors: Harry M. Meade, Newton, MA (US); Eszter Birck-Wilson, Ashland, MA (US); Daniel Pollock, Medway, MA (US)

Correspondence Address: GTC BIOTHERAPEUTICS, INC., 175 CROSSING BOULEVARD, SUITE 410, FRAMINGHAM, MA, 01702, US

(21) Appl. No.: 10/722,903

(22) Filed: Nov. 26, 2003

Related U.S. Application Data
(60) Provisional application No. 60/429,606, filed on Nov. 27, 2002.

(51) Int. Cl. A01K 67/027; C12P 21/00

(52) U.S. Cl. 800/7

(57) ABSTRACT

The invention features methods of producing an antibody in the milk of a transgenic mammal. The methods include providing a transgenic mammal whose somatic and germ cells comprise a sequence encoding an exogenous heavy chain variable region or antigen binding fragment thereof, at least one heavy chain constant region, or a fragment thereof, and a hinge region, operably linked to a promoter which directs expression in mammary epithelial cells, wherein said hinge region has been altered from the hinge region normally associated with the heavy chain constant region. The invention also features transgenic mammals, methods of producing these mammals, compositions comprising such antibodies, and nucleic acids encoding the antibodies.

### FIELD OF THE INVENTION

The present invention provides a method of producing antibodies in the milk of a transgenic mammal. The method includes providing a transgenic mammal whose somatic and germ cells have a sequence encoding at least a heavy and a light chain and at least one hinge region, wherein the hinge region has been altered from the hinge region normally associated with the heavy chain constant region to improve stability and folding properties of the resultant recombinant antibody.

### BACKGROUND OF THE INVENTION

IgG is the most abundant isotype of antibody in the serum of human adults, constituting approximately 80% of the total serum immunoglobulin. IgG is a monomeric molecule having a tetrameric structure consisting of two P_{U} heavy immunoglobulin chains and two (P₂ or S_{E}) light immunoglobulin chains. The heavy and light immunoglobulin chains are generally inter-connected by disulfide bonds. The antibody further includes a hinge region rich in proline residues, which confers segmental flexibility to the molecule. IgG demonstrates numerous biological functions, including agglutination of antigen, opsonization, antibody-dependent cell-mediated cytotoxicity, passage through the placenta, activation of complement, neutralization of toxins, immobilization of bacteria, and neutralization of viruses.

Due to their lack of effector function, IgG4 antibodies can be used as therapeutic agents. Unfortunately, IgG4 antibodies have the property of being “unstable” during acid treatment or on non-reducing polyacrylamide gel electrophoresis (PAGE), and can result in an 80 kDa protein (also known as a “half molecule”). The half molecule results if there is no disulfide bond linking the two heavy chains together.

Production of IgG4 in tissue culture has met with varied success. Depending upon cell lines, the percentage of “half molecule” IgG4 can vary between 5 and 25%. One of the problems in producing the IgG4 molecule is that there is no convenient method for separating the half molecule forms from whole IgG4 molecules. Many production facilities simply accept that there will be varying levels of the contaminating “half molecule” generated in the process.

### SUMMARY OF THE INVENTION

The present invention is based, in part, on the discovery that the production of antibodies in the milk of transgenic animals can result in up to 50% of the antibodies produced being in half molecule form, and that by modifying the hinge region of such antibodies, increased levels of assembled antibodies are obtained in the milk of such animals. Although not wishing to be bound by theory, the increased levels of half molecules found in the milk of transgenic animals may be due, in part, to the mammary gland being unable to permit proper folding and/or disulfide bond formation between heavy chains of an antibody while still providing efficient secretion. By modifying the hinge region of such antibodies, decreased levels of half molecules are obtained.

Thus, in one aspect, the invention features a method of producing antibodies in the milk of a transgenic mammal. The method includes providing a transgenic mammal whose somatic and germ cells have a sequence encoding an exogenous heavy chain variable region or antigen binding fragment thereof, at least one heavy chain constant region, or a fragment thereof, and a hinge region, operably linked to a promoter which directs expression in mammary epithelial cells, wherein the hinge region has been altered from the hinge region normally associated with the heavy chain constant region.

In one embodiment, at least 70%, 75%, 80%, 90%, or 95% of the antibodies present in the milk are in assembled form. In another embodiment, the somatic and germ cells of the transgenic mammal further include a sequence encoding a light chain variable region, or antigen binding fragment thereof, and a light chain constant region, or functional fragment thereof, operably linked to a promoter which directs expression in mammary epithelial cells.

In other embodiments, the method can include a step of obtaining milk from the transgenic mammal to provide an antibody composition. Further, the method can include the step of purifying the exogenous antibody from the milk.

The promoter used can be any promoter known in the art which directs expression in mammary epithelial cells, e.g. casein promoters, lactalbumin promoters, beta lactoglobulin promoters or whey acid protein promoters. In a preferred embodiment, the transgenic animal can be, e.g., cows, goats, mice, rats, sheep, pigs and rabbits.

The antibody can be any antibody from any antibody class, e.g. IgA, IgD, IgM, IgE or IgG, or fragments thereof. In a preferred embodiment, the antibody is an IgG antibody, e.g., an IgG1, IgG2, IgG3, or IgG4 antibody. In another preferred embodiment, the antibody is an IgG4 antibody.

Various alterations in the hinge region of the antibody are contemplated by the present invention. For example, in one embodiment, all or a portion of the hinge region of the antibody is modified. In another embodiment, all or a portion of the hinge region of the antibody is replaced, e.g. replaced with a hinge region or portion thereof which differs from the hinge region normally associated with the heavy chain constant and/or variable region. In a preferred embodiment, the hinge region of the antibody having a heavy chain constant region or portion thereof of an IgG antibody can be replaced with the hinge region, or portion thereof, of an antibody other than an IgG antibody. For example, the hinge region, or portion thereof, of an IgG antibody, e.g. an IgG1, IgG2, IgG3, or IgG4 antibody, can be replaced with hinge region or portion derived from an IgA, IgD, IgM, IgE antibody. In another embodiment, the hinge region, or portion thereof, of an antibody having a heavy chain constant region or portion thereof of an IgG antibody, e.g. an IgG1, IgG2, IgG or IgG4 antibody can be replaced with a hinge region or portion thereof derived from another IgG antibody, e.g. the hinge region of an IgG1, IgG2, IgG3 or IgG4 antibody can be replaced with a hinge derived from another subclass of IgG. In still another preferred embodiment, the hinge region of the antibody having a heavy chain constant region of an IgG4 antibody can be replaced with a hinge region derived from an IgG1, IgG2 or IgG3.

In still another embodiment, the hinge region has been modified such that at least one of the nucleic acid residues of the nucleic acid sequence encoding the hinge region of the antibody differs from the naturally occurring nucleic acid sequence of the hinge region normally associated with the heavy chain constant region of the antibody. In another embodiment, the amino acid sequence of the hinge region of the antibody differs from the amino acid sequence of the hinge region naturally occurring with the heavy chain constant region of the antibody by at least one amino acid residue.

In a preferred embodiment, the hinge region has been modified such that one or more amino acids of the hinge region naturally associated with the heavy chain constant region are substituted with an amino acid corresponding to that position in a hinge region associated with a heavy chain constant region of an antibody of a different class or subclass. Preferably, the heavy chain constant region of the antibody being produced is from an IgG antibody and the hinge region is substituted with 1 or more amino acids of the hinge region an IgA, IgD, IgM or IgE antibody. In another preferred embodiment, the heavy chain constant region of the antibody being produced is from an IgG antibody, e.g., an IgG4 antibody, and the hinge region is substituted with one or more amino acids of a hinge region of an antibody of a different subclass, e.g., of an IgG1, IgG2 and IgG3 antibody.

In another embodiment, at least one amino acid in the hinge region other than a cysteine residue can be replaced with a cysteine residue. Modifications can include altering at least one glycosylation site of the antibody, e.g. in the heavy chain or light chain, or in the hinge region of the heavy chain of the antibody.

In another embodiment, the heavy chain constant region of the antibody being produced is from an IgG4 antibody, and a serine residue of the hinge region can be replaced with a proline residue. For example, a serine residue at amino acid number 241 of the hinge region can be replaced with a proline residue.

The antibody can be, for example, chimeric, human, or a humanized antibody, or fragments thereof.

In another embodiment, the milk of the transgenic mammal is essentially free from the half molecule form of the exogenous antibody. Preferably, the ratio of assembled exogenous antibody to half forms of the antibody present in the milk of a transgenic mammal are at least 2:1, 3:1, 4:1, 5:1, 6:1, 7:1, 8:1, 9:1, 10:1, or greater (e.g., 20:1).

In another aspect, the invention features a method of producing a transgenic mammal whose somatic and germ cells include a modified antibody coding sequence, wherein the modified antibody coding sequence encodes an antibody molecule or portion thereof having an altered hinge region. The method includes the step of introducing into a mammal a construct, which includes a sequence encoding an exogenous heavy chain variable region or antigen binding fragment thereof, at least one heavy chain constant region or fragment thereof, and a hinge region, operably linked to a promoter which directs expression in mammary epithelial cells, wherein the hinge region has been altered from the hinge region normally associated with the heavy chain constant region of the antibody being produced. In one embodiment, the hinge region has been altered such that at least 70%, 75%, 80%, 85%, 90%, 95% of the exogenous antibodies present in the milk of the transgenic mammal are in assembled form. In another embodiment, the construct includes a sequence encoding a light chain variable region or antigen binding fragment thereof and a light chain constant region or functional fragment thereof, operably linked to a promoter that directs expression in mammary epithelial cells.

The promoter used can be any promoter known in the art which directs expression in mammary epithelial cells, e.g. casein promoters, lactalbumin promoters, beta lactoglobulin promoters or whey acid protein promoters. In a preferred embodiment, the transgenic animal can be, e.g., cows, goats, mice, rats, sheep, pigs and rabbits.

The antibody can be any antibody from any antibody class, e.g. IgA, IgD, IgM, IgE or IgG, or fragments thereof. In a preferred embodiment, the antibody is an IgG antibody, e.g., an IgG1, IgG2, IgG3, or IgG4 antibody. In another preferred embodiment, the antibody is an IgG4 antibody.

Various alterations in the hinge region of the antibody are contemplated by the present invention. For example, in one embodiment, all or a portion of the hinge region of the antibody is modified. In another embodiment, all or a portion of the hinge region of the antibody is replaced, e.g. replaced with a hinge region or portion thereof which differs from the hinge region normally associated with the heavy chain constant and/or variable region. In a preferred embodiment, the heavy chain constant region or portion thereof is from an IgG and hinge region of the antibody can be replaced with the hinge region, or portion thereof, of an antibody other than an IgG antibody. For example, the hinge region, or portion thereof, of an IgG antibody, e.g. an IgG1, IgG2, IgG3, or IgG4 antibody, can be replaced with hinge region or portion derived from an IgA, IgD, IgM, IgE antibody. In another embodiment, the hinge region, or portion thereof, of an antibody having a heavy chain constant region or portion thereof of an IgG antibody, e.g. an IgG1, IgG2, IgG or IgG4 antibody can be replaced with a hinge region or portion thereof derived from another IgG antibody, e.g. the hinge region of an IgG1, IgG2, IgG3 or IgG4 antibody can be replaced with a hinge derived from another subclass of IgG. In still another preferred embodiment, the hinge region of the antibody having a heavy chain constant region of an IgG4 antibody can be replaced with a hinge region derived from an IgG1, IgG2 or IgG3.

In still another embodiment, the hinge region has been modified such that at least one of the nucleic acid residues of the nucleic acid sequence encoding the hinge region of the antibody differs from the naturally occurring nucleic acid sequence of the hinge region normally associated with the heavy chain constant region of the antibody. In another embodiment, the amino acid sequence of the hinge region of the antibody differs from the amino acid sequence of the hinge region of the naturally occurring with the heavy chain constant region of the antibody by at least one amino acid residue.

In a preferred embodiment, the hinge region has been modified such that one or more amino acids of the hinge region naturally associated with the heavy chain constant region are substituted with an amino acid corresponding to that position in a hinge region associated with a heavy chain constant region of an antibody of a different class or subclass. Preferably, the heavy chain constant region of the antibody being produced is from an IgG antibody and the hinge region is substituted with 1 or more amino acids of the hinge region an IgA, IgD, IgM or IgE antibody. In another embodiment, the heavy chain constant region of the antibody being produced is from an IgG antibody, e.g., an IgG4 antibody, and the hinge region is substituted with one or more amino acids of a hinge region of an antibody of a different class, e.g., of an IgG1, IgG2 and IgG3 antibody.

In another embodiment, at least one amino acid in the hinge region other than a cysteine residue can be replaced with a cysteine residue. Modifications can include altering at least one glycosylation site of the antibody, e.g. in the heavy chain or light chain, or in the hinge region of the heavy chain of the antibody.

In another embodiment, the heavy chain constant region of the antibody being produced is from an IgG4 antibody, and a serine residue of the hinge region can be replaced with a proline residue. For example, a serine residue at amino acid number 241 of the hinge region of an IgG4 antibody can be replaced with a proline residue.

The antibody can be, for example, chimeric, human, or a humanized antibody, or fragments thereof.

In another embodiment, the milk of the transgenic mammal is essentially free from the half molecule form of the exogenous antibody. Preferably, the ratio of assembled exogenous antibody to half forms of the antibody present in the milk of a transgenic mammal are at least 2:1, 3:1, 4:1, 5:1, 6:1, 7:1, 8:1, 9:1, 10:1, or greater (e.g., 20:1). In preferred embodiments, the hinge region is altered such that at least 70%, 75%, 80%, 85%, 90%, 95% of the exogenous antibodies present in the milk of the transgenic mammal are in assembled form.

The present invention contemplates all manners known to those of skill in the art for introducing antibody coding sequences into transgenic animals. For example, coding sequences encoding portions of antibodies, e.g. heavy chain variable regions, light chain variable regions, heavy chain constant regions, light chain constant regions, etc., can be introduced as separate constructs, under the control of separate promoters, e.g., separate promoters which direct mammary epithelial cell expression. The separate promoters can be the same type of mammary epithelial cell promoters (e.g., both constructs include a casein promoter) or a different type of mammary epithelial cell promoter (e.g., one construct includes a casein promoter and the other a β-lactoglobulin promoter). Accordingly, in a related embodiment, the present invention provides a method of producing a transgenic mammal capable of expressing an assembled exogenous antibody or portion thereof in its milk, which includes the steps of introducing into a mammal a construct which includes a sequence encoding a light chain of exogenous antibody linked to a promoter which directs expression in mammary epithelial cells and introducing into the mammal a construct comprising a sequence encoding a mutagenized heavy chain of the exogenous antibody or a portion thereof linked to a promoter which directs expression in mammary epithelial cells. In another embodiment, the construct includes a sequence encoding a mutagenized heavy chain and a sequence encoding a light chain variable region or antigen binding fragment thereof and a light chain constant region or functional fragment thereof. The sequence encoding the mutagenized heavy chain and the sequence encoding the light chain or portion thereof may be operably linked to different promoters which direct expression in mammary epithelial cells, or can be under control of the same promoter. For example, the modified antibody coding sequence can be polycistronic, e.g., the heavy chain coding sequence and the light chain coding sequence can have an internal ribosome entry site (IRES) between them. When under the control of separate promoters, the promoters can be under the control of the same type of mammary epithelial cell promoter (e.g., both sequences are under the control of a β-casein promoter) or each is under the control of a different type of mammary epithelial promoter (e.g., one sequence is under the control of a β-casein promoter and the other is under the control of a β-lactoglobulin promoter).

In another embodiment, the invention provides a method of producing a transgenic mammal capable of expressing an assembled exogenous antibody in its milk, which includes the steps of providing a cell from a transgenic mammal whose germ and somatic cells include a sequence encoding a light chain of an exogenous antibody operably linked to a promoter which directs expression in mammary epithelial cells and introducing into the cell a construct comprising a sequence encoding a mutagenized heavy chain of the exogenous antibody or a portion thereof operably linked to a promoter which directs expression in mammary epithelial cells, wherein the heavy chain, or portion thereof includes a hinge region which has been altered from the hinge region normally associated with the heavy chain constant region. In still another embodiment, the invention provides a method of producing a transgenic mammal capable of expressing an assembled exogenous antibody in its milk, which includes the steps of providing a cell from a transgenic mammal whose germ and somatic cells include a sequence encoding a mutagenized heavy chain or portion thereof of an exogenous antibody, operably linked to a promoter which directs expression in mammary epithelial cells, and introducing into the cell a construct comprising a sequence encoding a light chain of an exogenous antibody operably linked to a promoter which directs expression in mammary epithelial cells.

In yet another aspect, the present invention features a transgenic mammal capable of expressing an exogenous antibody in milk, wherein the somatic and germ cells of the transgenic mammal include a modified antibody coding sequence encoding an exogenous heavy chain variable region or antigen binding fragment thereof, at least one heavy chain constant region or a fragment thereof, and a hinge region operably linked to a promoter which directs expression in mammary epithelial cells, wherein the hinge region has been altered from the hinge region normally associated with the heavy chain constant region of the antibody being produced.

The promoter used can be any promoter known in the art which directs expression in mammary epithelial cells, e.g. casein promoters, lactalbumin promoters, beta lactoglobulin promoters or whey acid protein promoters. In a preferred embodiment, the transgenic animal can be, e.g., cows, goats, mice, rats, sheep, pigs and rabbits.

The antibody can be any antibody from any antibody class, e.g. IgA, IgD, IgM, IgE or IgG, or fragments thereof. In a preferred embodiment, the antibody is an IgG antibody, e.g., an IgG1, IgG2, IgG3, or IgG4 antibody. In another preferred embodiment, the antibody is an IgG4 antibody.

Various alterations in the hinge region of the antibody are contemplated by the present invention. For example, in one embodiment, all or a portion of the hinge region of the antibody is modified. In another embodiment, all or a portion of the hinge region of the antibody is replaced, e.g. replaced with a hinge region or portion thereof which differs from the hinge region normally associated with the heavy chain constant and/or variable region. In a preferred embodiment, the hinge region of the antibody having a heavy chain constant region or portion thereof of an IgG antibody can be replaced with the hinge region, or portion thereof, of an antibody other than an IgG antibody. For example, the hinge region, or portion thereof, of an IgG antibody, e.g. an IgG1, IgG2, IgG3, or IgG4 antibody, can be replaced with hinge region or portion derived from an IgA, IgD, IgM, IgE antibody. In another embodiment, the hinge region, or portion thereof, of an antibody having a heavy chain constant region or portion thereof of an IgG antibody, e.g. an IgG1, IgG2, IgG or IgG4 antibody can be replaced with a hinge region or portion thereof derived from another IgG antibody, e.g. the hinge region of an IgG1, IgG2, IgG3 or IgG4 antibody can be replaced with a hinge derived from another subclass of IgG. In still another preferred embodiment, the hinge region of the antibody having a heavy chain constant region of an IgG4 antibody can be replaced with a hinge region derived from an IgG1, IgG2 or IgG3.

In still another embodiment, the hinge region has been modified such that at least one of the nucleic acid residues of the nucleic acid sequence encoding the hinge region of the antibody differs from the naturally occurring nucleic acid sequence of the hinge region normally associated with the heavy chain constant region of the antibody. In another embodiment, the amino acid sequence of the hinge region of the antibody differs from the amino acid sequence of the hinge region of the naturally occurring with the heavy chain constant region of the antibody by at least one amino acid residue.

In a preferred embodiment, the hinge region has been modified such that one or more amino acids of the hinge region naturally associated with the heavy chain constant region are substituted with an amino acid corresponding to that position in a hinge region associated with a heavy chain constant region of an antibody of a different class or subclass. Preferably, the heavy chain constant region of the antibody being produced is from an IgG antibody and the hinge region is substituted with 1 or more amino acids of the hinge region an IgA, IgD, IgM or IgE antibody. More preferably, the heavy chain constant region of the antibody being produced is from an IgG antibody, e.g., an IgG4 antibody, and the hinge region is substituted with one or more amino acids of a hinge region of an antibody of a different class, e.g., of an IgG1, IgG2 and IgG3 antibody.

In another embodiment, at least one amino acid in the hinge region other than a cysteine residue can be replaced with a cysteine residue. Modifications can include altering at least one glycosylation site of the antibody, e.g. in the heavy chain or light chain, or in the hinge region of the heavy chain of the antibody.

In another embodiment, the heavy chain constant region of the antibody being produced is from an IgG4 antibody, and a serine residue of the hinge region can be replaced with a proline residue. For example, a serine residue at amino acid number 241 of the hinge region can be replaced with a proline residue.

The antibody can be, for example, chimeric, human, or a humanized antibody, or fragments thereof.

In another embodiment, the milk of the transgenic mammal is essentially free from the half molecule form of the exogenous antibody. Preferably, the ratio of assembled exogenous antibody to half forms of the antibody present in the milk of a transgenic mammal are at least 2:1, 3:1, 4:1, 5:1, 6:1, 7:1, 8:1, 9:1, 10:1, or greater (e.g., 20:1).

In preferred embodiments, the hinge region is altered such that at least 70%, 75%, 80%, 85%, 90%, 95% of the exogenous antibodies present in the milk of the transgenic mammal are in assembled form. In another embodiment, the modified antibody coding sequence further includes a sequence encoding a light chain variable region or antigen binding fragment thereof and a light chain constant region or functional fragment thereof. The light chain variable region or antigen binding fragment thereof and light chain constant region or functional fragment thereof may be operably linked to a promoter which directs expression in mammary epithelial cells, or under control of the same promoter as the sequence encoding the exogenous heavy chain variable region, heavy chain constant region (or portions thereof), and hinge region. For example, the modified antibody coding sequence can be polycistronic, e.g., the heavy chain coding sequence and the light chain coding sequence can have an internal ribosome entry site (IRES) between them.

In yet another aspect, the invention provides a composition which includes a milk component and an antibody component described herein. Preferably, at least 70%, 75%, 80%, 85%, 90%, 95% of the exogenous antibodies are in assembled form. In another embodiment, the hinge region has been altered such that at least 70%, 75%, 80%, 85%, 90%, 95% of the exogenous antibodies present in the composition are in assembled form.

The antibody can be any antibody from any antibody class, e.g. IgA, IgD, IgM, IgE or IgG, or fragments thereof. In a preferred embodiment, the antibody is an IgG antibody, e.g., an IgG1, IgG2, IgG3, or IgG4 antibody. In another preferred embodiment, the antibody is an IgG4 antibody.

Various alterations in the hinge region of the antibody are contemplated by the present invention. For example, in one embodiment, all or a portion of the hinge region of the antibody is modified. In another embodiment, all or a portion of the hinge region of the antibody is replaced, e.g. replaced with a hinge region or portion thereof which differs from the hinge region normally associated with the heavy chain constant and/or variable region. In a preferred embodiment, the hinge region of the antibody having a heavy chain constant region or portion thereof of an IgG antibody can be replaced with the hinge region, or portion thereof, of an antibody other than an IgG antibody. For example, the hinge region, or portion thereof, of an IgG antibody, e.g. an IgG1, IgG2, IgG3, or IgG4 antibody, can be replaced with hinge region or portion derived from an IgA, IgD, IgM, IgE antibody. In another embodiment, the hinge region, or portion thereof, of an antibody having a heavy chain constant region or portion thereof of an IgG antibody, e.g. an IgG1, IgG2, IgG or IgG4 antibody can be replaced with a hinge region or portion thereof derived from another IgG antibody, e.g. the hinge region of an IgG1, IgG2, IgG3 or IgG4 antibody can be replaced with a hinge derived from another subclass of IgG. In still another preferred embodiment, the hinge region of the antibody having a heavy chain constant region of an IgG4 antibody can be replaced with a hinge region derived from an IgG1, IgG2 or IgG3.

In still another embodiment, the hinge region has been modified such that at least one of the nucleic acid residues of the nucleic acid sequence encoding the hinge region of the antibody differs from the naturally occurring nucleic acid sequence of the hinge region normally associated with the heavy chain constant region of the antibody. In another embodiment, the amino acid sequence of the hinge region of the antibody differs from the amino acid sequence of the hinge region of the naturally occurring with the heavy chain constant region of the antibody by at least one amino acid residue.

In a preferred embodiment, the hinge region has been modified such that one or more amino acids of the hinge region naturally associated with the heavy chain constant region are substituted with an amino acid corresponding to that position in a hinge region associated with a heavy chain constant region of an antibody of a different class or subclass. Preferably, the heavy chain constant region of the antibody being produced is from an IgG antibody and the hinge region is substituted with 1 or more amino acids of the hinge region an IgA, IgD, IgM or IgE antibody. More preferably, the heavy chain constant region of the antibody being produced is from an IgG antibody, e.g., an IgG4 antibody, and the hinge region is substituted with one or more amino acids of a hinge region of an antibody of a different class, e.g., of an IgG1, IgG2 and IgG3 antibody.

In another embodiment, at least one amino acid in the hinge region other than a cysteine residue can be replaced with a cysteine residue. Modifications can include altering at least one glycosylation site of the antibody, e.g. in the heavy chain or light chain, or in the hinge region of the heavy chain of the antibody.

In another embodiment, the heavy chain constant region of the antibody being produced is from an IgG4 antibody, and a serine residue of the hinge region can be replaced with a proline residue. For example, a serine residue at amino acid number 241 of the hinge region can be replaced with a proline residue.

The antibody can be, for example, chimeric, human, or a humanized antibody, or fragments thereof.

In another embodiment, the milk of the transgenic mammal is substantially free from the half molecule form of the exogenous antibody. Preferably, the ratio of assembled exogenous antibody to half forms of the antibody present in the milk of a transgenic mammal are at least 2:1, 3:1, 4:1, 5:1, 6:1, 7:1, 8:1, 9:1, 10:1, or greater (e.g., 20:1).

In another preferred embodiment, the composition is substantially free of the milk component, e.g., the milk component or components makes up less than 10%, 5%, 3%, 2%, 1%, 0.5%, 0.2% of the volume by weight. Examples of milk components include casein, lipids (e.g., soluble lipids and phospholipids), lactose and other small molecules (e.g., galactose, glucose), small peptides (e.g., microbial peptides, antimicrobial peptides) and other milk proteins (e.g., whey proteins such as β-lactoglobulin and α-lactalbumin, lactoferrin, and serum albumin).

In yet another aspect, the invention provides a nucleic acid which includes a sequence encoding a heavy chain variable region or antigen binding portion thereof and a heavy chain constant region or fragment thereof and a hinge region, operably linked to a promoter which directs expression in mammary epithelial cells, wherein the hinge region has been altered from the hinge region normally associated with the heavy chain constant region.

The promoter used can be any promoter known in the art which directs expression in mammary epithelial cells, e.g. casein promoters, lactalbumin promoters, beta lactoglobulin promoters or whey acid protein promoters. The heavy chain variable region or antigen binding portion thereof and heavy chain constant region or fragment thereof and hinge region can be from any antibody from any antibody class, e.g. IgA, IgD, IgM, IgE or IgG, or fragments thereof. In a preferred embodiment, the antibody is an IgG antibody, e.g., an IgG1, IgG2, IgG3, or IgG4 antibody. In another preferred embodiment, the antibody is an IgG4 antibody.

Various alterations in the hinge region are contemplated by the present invention. For example, in one embodiment, all or a portion of the hinge region is modified. In another embodiment, all or a portion of the hinge region is replaced, e.g. replaced with a hinge region or portion thereof which differs from the hinge region normally associated with the heavy chain constant and/or variable region. In a preferred embodiment, the hinge region of the antibody having a heavy chain constant region or portion thereof of an IgG antibody can be replaced with the hinge region, or portion thereof, of an antibody other than an IgG antibody. For example, the hinge region, or portion thereof, of an IgG antibody, e.g., an IgG1, IgG2, IgG3, or IgG4 antibody, can be replaced with hinge region or portion derived from an IgA, IgD, IgM, IgE antibody. In another embodiment, the hinge region, or portion thereof, of an antibody having a heavy chain constant region or portion thereof of an IgG antibody, e.g., an IgG1, IgG2, IgG or IgG4 antibody can be replaced with a hinge region or portion thereof derived from another IgG antibody, e.g., the hinge region of an IgG1, IgG2, IgG3 or IgG4 antibody can be replaced with a hinge derived from another subclass of IgG. In still another preferred embodiment, the hinge region of the antibody having a heavy chain constant region of an IgG4 antibody can be replaced with a hinge region derived from an IgG1, IgG2 or IgG3.

In still another embodiment, the hinge region has been modified such that at least one of the nucleic acid residues of the nucleic acid sequence encoding the hinge region of the antibody differs from the naturally occurring nucleic acid sequence of the hinge region normally associated with the heavy chain constant region. In another embodiment, the amino acid sequence of the hinge region differs from the amino acid sequence of the hinge region naturally occurring with the heavy chain constant region of the antibody by at least one amino acid residue.

In a preferred embodiment, the hinge region has been modified such that one or more amino acids of the hinge region naturally associated with the heavy chain constant region are substituted with an amino acid corresponding to that position in a hinge region associated with a heavy chain constant region of an antibody of a different class or subclass. Preferably, the heavy chain constant region of the antibody being produced is from an IgG antibody and the hinge region is substituted with 1 or more amino acids of the hinge region an IgA, IgD, IgM or IgE antibody. In another preferred embodiment, the heavy chain constant region of the antibody being produced is from an IgG antibody, e.g., an IgG4 antibody, and the hinge region is substituted with one or more amino acids of a hinge region of an antibody of a different class, e.g., of an IgG1, IgG2 and IgG3 antibody.

In another embodiment, at least one amino acid in the hinge region other than a cysteine residue can be replaced with a cysteine residue. Modifications can include altering at least one glycosylation site of the antibody, e.g. in the heavy chain or light chain, or in the hinge region of the heavy chain of the antibody.

In another embodiment, the heavy chain constant region of the antibody being produced is from an IgG4 antibody, and a serine residue of the hinge region can be replaced with a proline residue. For example, a serine residue at amino acid number 241 of the hinge region can be replaced with a proline residue.

The antibody can be, for example, chimeric, human, or a humanized antibody, or fragments thereof.

In some embodiments, the nucleic acid can be polycistronic, e.g., the heavy chain coding sequence and the light chain coding sequence can be under the control of the same promoter, e.g., by having an internal ribosome entry site (IRES) between them.

### BRIEF DESCRIPTION OF THE DRAWINGS

FIG. 1 Shows A Generalized Diagram of the Process of Creating Cloned Animals through Nuclear Transfer.

FIG. 2 Shows an Overview Of Analytics Performed With KMK917 With Regard To Hinge Region Modification.

FIG. 3A Shows an CEx-HPLC graph of an isolated KMK antibody sample.

FIG. 3B Shows an CEx-HPLC graph of an isolated KMK antibody sample.

FIG. 3C Shows an CEx-HPLC graph of an isolated KMK antibody sample.

FIG. 3D Shows an CEx-HPLC graph of an isolated KMK antibody sample.

FIG. 3E Shows an CEx-HPLC graph of an isolated KMK antibody sample.

FIG. 3F Shows an CEx-HPLC graph of an isolated KMK antibody sample.

FIG. 3G Shows an CEx-HPLC graph of an isolated KMK antibody sample.

FIG. 4A Shows an CEx-HPLC of KMK wild type sample±Endoglycosidase F treatment, wild type.

FIG. 4B Shows an CEx-HPLC of KMK wild type sample±Endoglycosidase F treatment, wild type.

FIG. 4 Cc Shows a CEx-HPLC of KMK wild type sample±Endoglycosidase F treatment, hinge and CH2 mutant.

FIG. 4D CEx-HPLC of KMK wild type sample±Endoglycosidase F treatment, hinge and CH2 mutant.

FIG. 5A Shows a CEx-HPLC graph of the Carbohydrate pattern of KMK917 1099/2010, wild type.

FIG. 5B Shows a CEx-HPLC graph of the Carbohydrate pattern of KMK917 2012/2014 hinge+Ch2 mutant.

FIG. 5C Shows a CEx-HPLC graph of the Carbohydrate pattern of KMK917, Full Scale

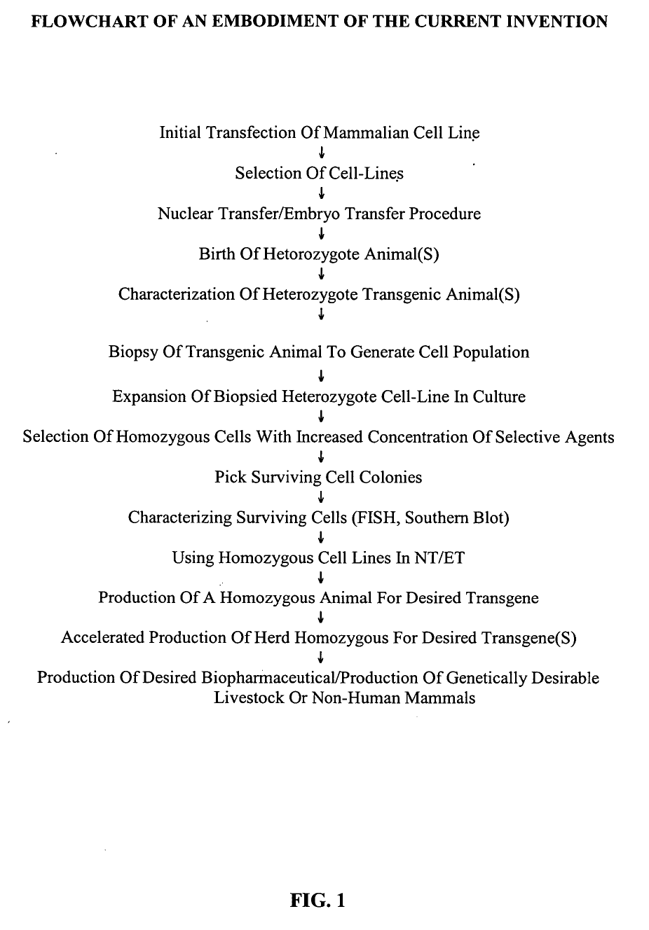

### DETAILED DESCRIPTION

The following abbreviations have designated meanings in the specification:

Abbreviation Key:

| | |
|---|---|
| Somatic Cell Nuclear Transfer | (SCNT) |
| Cultured Inner Cell Mass Cells | (CICM) |
| Nuclear Transfer | (NT) |
| Synthetic Oviductal Fluid | (SOF) |
| Fetal Bovine Serum | (FBS) |
| Polymerase Chain Reaction | (PCR) |
| Bovine Serum Albumin | (BSA) |
| High Pressure Liquid Chromatography | (HPLC) |

Explanation of Terms:
Bovine—Of or relating to various species of cows.
Caprine—Of or relating to various species of goats.
Cell Couplet—An enucleated oocyte and a somatic or fetal karyoplast prior to fusion and/or activation.
Cytocholasin-B—A metabolic product of certain fungi that selectively and reversibly blocks cytokinesis while not effecting karyokinesis.
Cytoplast—The cytoplasmic substance of eukaryotic cells.
Fusion Slide—A glass slide for parallel electrodes that are placed a fixed distance apart. Cell couplets are placed between the electrodes to receive an electrical current for fusion and activation.
Karyoplast—A cell nucleus, obtained from the cell by enucleation, surrounded by a narrow rim of cytoplasm and a plasma membrane.
Nuclear Transfer—or “nuclear transplantation” refers to a method of cloning wherein the nucleus from a donor cell is transplanted into an enucleated oocyte.
Ovine—of, relating to or resembling sheep.
Parthenogenic—The development of an embryo from an oocyte without the penetrance of sperm
Porcine—of, relating to or resembling swine or pigs
Reconstructed Embryo—A reconstructed embryo is an oocyte that has had its genetic material removed through an enucleation procedure. It has been “reconstructed” through the placement of genetic material of an adult or fetal somatic cell into the oocyte following a fusion event.
Selective Agent—Compounds, compositions, or molecules that can act as selection markers for cells in that they are capable of killing and/or preventing the growth of a living organism or cell not containing a suitable resistance gene. According to the current invention such agents include, without limitation, Neomycin, puromycin, zeocin, hygromycin, G418, gancyclovir and FIAU. Preferably, for the current invention increasing the dosage of the selective agent will kill all cell lines that only contain one integration site (e.g., heterozygous animals and/or cells).
Somatic Cell—Any cell of the body of an organism except the germ cells.
Somatic Cell Nuclear Transfer—Also called therapeutic cloning, is the process by which a somatic cell is fused with an enucleated oocyte. The nucleus of the somatic cell provides the genetic information, while the oocyte provides the nutrients and other energy-producing materials that are necessary for development of an embryo. Once fusion has occurred, the cell is totipotent, and eventually develops into a blastocyst, at which point the inner cell mass is isolated.
Transgenic Organism—An organism into which genetic material from another organism has been experimentally transferred, so that the host acquires the genetic information of the transferred genes in its chromosomes in addition to that already in its genetic complement.
Ungulate—of or relating to a hoofed typically herbivorous quadraped mammal, including, without limitation, sheep, swine, goats, cattle and horses.
Xenotransplantation—any procedure that involves the use of live cells, tissues, and organs from one animal source, transplanted or implanted into another animal species (typically humans) or used for clinical ex-vivo perfusion

### DETAILED DESCRIPTION OF THE INVENTION

The invention pertains to the production of antibodies in the milk of a transgenic mammal. Various aspects of the invention relate to antibodies and antibody fragments, methods of producing an antibody or fragments thereof in the milk of a transgenic mammal, and methods of producing a transgenic mammal whose somatic and germ cells include a modified antibody coding sequence. Nucleic acid sequences for expression of a modified antibody coding sequence in mammary epithelial cells are also provided.

In order that the present invention may be more readily understood, certain terms are defined. Definitions are set forth throughout the detailed description.

Antibodies and Fragments Thereof

As used herein, a “class” of antibodies refers to the five major isotypes of antibodies, including IgA, IgD, IgE, IgG, and IgM. A “subclass” of antibodies refers to the a subclassification of a given class of antibodies based on amino acid differences among members of the class, e.g., the class of antibodies designated IgG can be divided into the subclasses of, e.g., IgG1, IgG2, IgG3, and IgG4, and the class of antibodies designated as IgA can be divided into the subclasses of IgA1 and IgA2.

The term “antibody” refers to a protein comprising at least one, and preferably two, heavy (H) chain variable regions (abbreviated herein as VH), at least one and preferably two light (L) chain variable regions (abbreviated herein as VL), and at least one, preferably two heavy chain constant regions. The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The antibody can further include a light chain constant region, to thereby form a heavy and light immunoglobulin chains. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

The antibody can further include a hinge region, described in further detail below. As used herein, an “assembled” antibody is an antibody in which the heavy chains are associated with each other, e.g., interconnected by disulfide bonds. Each heavy chain hinge region includes at least one, and often several, cysteine residues. In the assembled antibody, the cysteine residues in the heavy chains are aligned so that disulphide bonds can be formed between the cysteine residues in the hinge regions covalently bonding the two heavy-light chain heterodimers together. Thus, fully assembled antibodies are bivalent in that they have two antigen binding sites. The term “antibody” (or “immunoglobulin”) as used herein, also refers to fragments of a full-length antibody, such as, e.g., a F(ab′)2 fragment, a bivalent fragment comprising two Fab fragments linked by a disulfide bridge at the hinge region. These antibody fragments are obtained using conventional techniques known to those with skill in the art, and the fragments are screened for utility in the same manner as are intact antibodies.

An “antigen-binding fragment” of an antibody (or “functional fragments”) refers to one or more portions of an antibody that retain the ability to specifically bind to an antigen. Examples of binding fragments encompassed within the term “antigen-binding fragment” of an antibody include one or more complementarities determining region (CDR).

As used herein, a “chimeric antibody heavy chain” refers to those antibody heavy chains having a portion of the antibody heavy chain, e.g., the variable region, at least 85%, preferably, 90%, 95%, 99% or more identical to a corresponding amino acid sequence in an antibody heavy chain from a particular species, or belonging to a particular antibody class or type, while the remaining segment of the antibody heavy chain (e.g., the constant region) being substantially identical to the corresponding amino acid sequence in another antibody molecule. For example, the heavy chain variable region has a sequence substantially identical to the heavy chain variable region of an antibody from one species (e.g., a “donor” antibody, e.g., a rodent antibody), while the constant region is substantially identical to the constant region of another species antibody (e.g., an “acceptor” antibody, e.g., a human antibody). The donor antibody can be an in vitro generated antibody, e.g., an antibody generated by phage display.

The term “humanized” or “CDR-grafted” light chain variable region refers to an antibody light chain comprising one or more CDR's, or having an amino acid sequence which differs by no more than 1 or 2 amino acid residues to a corresponding one or more CDR's from one species, or antibody class or type, e.g., a “donor” antibody (e.g., a non-human (usually a mouse or rat) immunoglobulin, or an in vitro generated immunoglobulin); and a framework region having an amino acid sequence about 85% or higher, preferably 90%, 95%, 99% or higher identical to a corresponding part of an acceptor antibody framework from a different species, or antibody class or type, e.g., a naturally-occurring immunoglobulin framework (e.g., a human framework) or a consensus framework. In some embodiments, the framework region includes at least about 60, and more preferably about 70 amino acid residues identical to those in the acceptor antibody light chain variable region framework, e.g., a naturally-occurring antibody framework (e.g., a human framework) or a consensus framework.

A “heterologous antibody” or “exogenous antibody” is an antibody that normally is not produced by the mammal, or is not normally produced in the mammary gland (e.g., an antibody only present in serum), or is produced in the mammary gland but the level of expression is augmented or enhanced in its production.

Any of the antibodies described herein, e.g., chimeric, humanized or human antibodies, can include further modifications to their sequence. E.g., the sequence can be modified by addition, deletion or substitution, e.g., a conservative substitution.

Antibody Hinge Regions

The methods of the present invention involve, for example, producing antibodies in the milk of a transgenic animal, wherein the hinge region has been altered from the hinge region normally associated with the heavy chain constant region of the antibody. Such a constant region is also referred to herein as “a mutagenized heavy chain constant region.” The term “normally associated” refers to the association between the hinge region and the heavy chain constant region in a naturally-occurring antibody. The term “naturally-occurring” as used herein refers to the fact that the antibody can be found in nature, e.g. in a natural organism. For example, an antibody or fragment thereof that is present in a natural organism, and which has not been intentionally modified by man, is naturally-occurring. The term also refers to the association between a hinge region and at least a portion of a heavy chain constant region (e.g., a CH1 region) of an antibody where that portion of the heavy chain constant region and the hinge region are found “naturally occurring” together in an antibody. This term is not limited to heavy chain constant regions only as found in nature. The constant chain region can include modifications, e.g., a substitution, insertion, or deletion of one or more amino acids. Examples of IgG hinge regions and heavy chain constant regions (or portions thereof) which are normally associated” with each other include: a hinge region of an IgG1 antibody and a heavy chain constant region (or portion thereof) of the same IgG1 antibody; a hinge region of an IgG2 antibody and a heavy chain constant region (or portion thereof) of the same IgG2 antibody; a hinge region of an IgG3 antibody and a heavy chain constant region (or portion thereof) of the same IgG3 antibody; and a hinge region of an IgG4 antibody and a heavy chain constant region (or portion thereof) of the same IgG4 antibody. These examples are non-limiting and such terminology is also applicable to other classes of antibodies.

As used herein, the “hinge region” of an antibody refers to a stretch of peptide sequence between the CH1 and CH2 domains of an antibody. Hinge regions occur between Fab and Fc portions of an antibody. Hinge regions are generally encoded by unique exons, and contain disulfide bonds that link the two heavy chain fragments of the antibody. See Paul et al., Fundamental Immunology, 3^{rd} Ed. (1993). The amino acid sequence of a hinge region can be generally rich in proline, serine, and threonine residues. For example, the extended peptide sequences between the CH1 and CH2 domains of IgG, IgD, and IgA are rich in prolines. IgM and IgE antibodies include a domain of about 110 amino acids that possesses hinge-like features (Ruby, J., Immunology (1992)), and are included in the term “hinge region” as used herein.

The amino acid sequence of the hinge region can include cysteine residues. Cysteine residues play a role in the formation of interchain disulfide bonds. Depending upon the class of the antibody, there can be between 2 and 11 inter-heavy chain disulfide bonds in the hinge region of the antibody. These disulfide bonds are responsible for holding together the two parts of the complete antibody molecule. The hinge regions of various classes and subclasses of antibodies are known in the art.

Alterations

Standard molecular biology techniques can be used to provide antibodies having altered hinge regions. These techniques can be used to create alterations, e.g., deletions, insertions, or substitutions, in the known amino acid sequence of the antibody hinge region (or other portions of the antibody sequence). The term “altered” refers to any change made within the hinge region of an antibody, or portion thereof. Such alterations include, but are not limited to, deletions, insertions, and replacements/substitutions of one or more or all of the amino acids of the hinge region. The skilled practitioner will appreciate that any suitable technique, such as directed or random mutagenesis techniques, can be used to provide specific sequences or mutations in the hinge region. Such techniques can also be used to alter other regions of the antibody, e.g., the heavy chain and/or light chain constant and/or variable region.

For example, oligonucleotide-mediated mutagenesis is a useful method for preparing substitution, deletion, and insertion variants of DNA, see, e.g., Adelman et al., (DNA 2:183, 1983). Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. USA, 75: 5765[1978]).

For example, in one embodiment, the hinge region of the antibody, or a fragment of the hinge region, is replaced by another hinge region, or fragment of the hinge region, from a different antibody, e.g., a different class or subclass of antibody. In a preferred embodiment, the IgG4 hinge region is replaced with a hinge region from a different subclass, e.g., an IgG2 hinge region. Such replacement can be performed, for example, using oligonucleotide-mediated mutagenesis, with an oligo that encodes an exon containing the IgG2 hinge region. In another embodiment, a single amino acid within a hinge region, e.g., an IgG4 hinge region, is replaced with a different amino acid, e.g. an amino acid found in a corresponding position in the hinge region of a different subclass, e.g., an amino acid of an IgG2 hinge region. For example, a serine found at amino acid 241 can be replaced with a proline (as found in a corresponding position in an IgG2 hinge region). Oligonucleotide-mediated mutagenesis can be used to make the replacement, using an oligo which causes the amino acid change (e.g. oligo S241P). In yet another embodiment, a glycosylation site of the antibody, e.g. an IgG4 antibody, is altered, e.g., is altered such that it no longer serves as a glycosylation site. For example, an N-linked glycosylation site could be altered such that an asparagine is changed to a glutamine. Oligonucleotide-mediated mutagenesis can also be used to effectuate this alteration, e.g. by using an oligo which causes the amino acid change.

Another example of a method for providing altered proteins, cassette mutagenesis, is based on the technique described by Wells et al. (Gene, 34:315[1985]). The starting material is a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid thus contains the mutated desired protein subunit DNA sequence.

It is further contemplated by the present invention that random mutagenesis of DNA which encodes an antibody or fragment thereof can also be used to create antibodies having altered hinge regions. Useful methods include, but are not limited to, PCR mutagenesis, saturation mutagenesis, and the creation and use of a set of degenerate oligonucleotide sequences. These methods are known.

Transgenic Mammals

As used herein, a “transgenic animal” is a non-human animal in which one or more, and preferably essentially all, of the cells of the animal contain a heterologous nucleic acid introduced by way of human intervention, such as by transgenic techniques known in the art. A transgene can be introduced into the cell, directly or indirectly, by introduction into a precursor of the cell, by way of deliberate genetic manipulation, such as by microinjection or by infection with a recombinant virus.

The term “transgene” means a nucleic acid sequence (encoding, e.g., one or more antibody polypeptides or portions thereof), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene). A transgene can include one or more transcriptional regulatory sequences and any other nucleic acid, such as introns, that may be necessary for optimal expression and secretion of the selected nucleic acid encoding the antibody, e.g., in a mammary gland, all operably linked to the selected antibody nucleic acid, and may include an enhancer sequence and/or an insulator sequence. The antibody sequence can be operatively linked to a tissue specific promoter, e.g., mammary gland specific promoter sequence that results in the secretion of the protein in the milk of a transgenic mammal.

As used herein, the term “transgenic cell” refers to a cell containing a transgene. Mammals are defined herein as all animals, excluding humans that have mammary glands and produce milk. Any non-human mammal can be utilized in the present invention. Preferred non-human mammals are ruminants, e.g., cows, sheep, camels or goats. Additional examples of preferred non-human animals include oxen, horses, llamas, and pigs. For example, methods of producing transgenic goats are known in the art. The transgene can be introduced into the germline of a goat by microinjection as described, for example, in Ebert et al. (1994) Bio/Technology 12:699, hereby incorporated by reference. The specific line(s) of any animal used to practice this invention are selected for general good health, good embryo yields, good pronuclear visibility in the embryo, and good reproductive fitness. In addition, the haplotype is a significant factor.

Methods for generating non-human transgenic mammals are known in the art. Such methods can involve introducing DNA constructs into the germ line of a mammal to make a transgenic mammal. For example, one or several copies of the construct may be incorporated into the genome of a mammalian embryo by standard transgenic techniques. In addition, non-human transgenic mammals can be produced using a somatic cell as a donor cell. The genome of the somatic cell can then be inserted into an oocyte and the oocyte can be fused and activated to form a reconstructed embryo. For example, methods of producing transgenic animals using a somatic cell are described in PCT Publication WO 97/07669; Baguisi et al. NATURE BIOTECH., vol. 17 (1999), 456-461; Campbell et al., NATURE, vol. 380 (1996), 64-66; Cibelli et al., SCIENCE, vol. 280 (1998); Kato et al., SCIENCE, vol. 282 (1998), 2095-2098; Schnieke et al., SCIENCE, vol. 278. (1997), 2130-2133; Wakayama et al., NATURE, vol. 394 (1998), 369-374; Well et al., BIOL. REPROD., vol. 57 (1997):385-393.

Transfected Cell Lines

Genetically engineered cell lines can be used to produce a transgenic animal. A genetically engineered construct can be introduced into a cell via conventional transformation or transfection techniques. As used herein, the terms “transfection” and “transformation” include a variety of techniques for introducing a transgenic sequence into a host cell, including calcium phosphate or calcium chloride co-precipitation, DEAE-dextrane-mediated transfection, lipofection, or electroporation. In addition, biological vectors, e.g., viral vectors can be used as described below. Suitable methods for transforming or transfecting host cells can be found in Sambrook et al., Molecular Cloning: A Laboratory Manuel, 2^{nd} ed., Cold Spring Harbor Laboratory, (Cold Spring Harbor Laboratory Press, Cold Spring Harbor, N.Y., 1989), and other suitable laboratory manuals.

Two useful approaches are electroporation and lipofection. Brief examples of each are described below.

The DNA construct can be stably introduced into a donor cell line by electroporation using the following protocol: somatic cells, e.g., fibroblasts, e.g., embryonic fibroblasts, are re-suspended in PBS at about 4×10⁶ cells/ml. Fifty micrograms of linearized DNA is added to the 0.5 ml cell suspension, and the suspension is placed in a 0.4 cm electrode gap cuvette (Biorad). Electroporation is performed using a Biorad Gene Pulser electroporator with a 330 volt pulse at 25 mA, 1000 microFarad and infinite resistance. If the DNA construct contains a Neomyocin resistance gene for selection, neomyocin resistant clones are selected following incubation with 350 microgram/ml of G418 (GibcoBRL) for 15 days.

The DNA construct can be stably introduced into a donor somatic cell line by lipofection using a protocol such as the following: about 2×10⁵ cells are plated into a 3.5 cm diameter well and transfected with 2 micrograms of linearized DNA using LipfectAMINE™ (GibcoBRL). Forty-eight hours after transfection, the cells are split 1:1000 and 1:5000 and, if the DNA construct contains a neomyosin resistance gene for selection, G418 is added to a final concentration of 0.35 mg/ml. Neomyocin resistant clones are isolated and expanded for cryopreservation as well as nuclear transfer.

DNA Constructs

A cassette which encodes a heterologous protein can be assembled as a construct which includes a promoter for a specific tissue, e.g., for mammary epithelial cells, e.g., a casein promoter, e.g., a goat beta casein promoter, a milk-specific signal sequence, e.g., a casein signal sequence, e.g., a β-casein signal sequence, and a DNA encoding the heterologous protein.

The construct can also include a 3′ untranslated region downstream of the DNA sequence coding for the non-secreted protein. Such regions can stabilize the RNA transcript of the expression system and thus increases the yield of desired protein from the expression system. Among the 3′ untranslated regions useful in the constructs for use in the invention are sequences that provide a poly A signal. Such sequences may be derived, e.g., from the SV40 small t antigen, the casein 3′ untranslated region or other 3′ untranslated sequences well known in the art. In one aspect, the 3′ untranslated region is derived from a milk specific protein. The length of the 3′ untranslated region is not critical but the stabilizing effect of its poly A transcript appears important in stabilizing the RNA of the expression sequence.

Optionally, the construct can include a 5′ untranslated region between the promoter and the DNA sequence encoding the signal sequence. Such untranslated regions can be from the same control region from which promoter is taken or can be from a different gene, e.g., they may be derived from other synthetic, semi-synthetic or natural sources. Again their specific length is not critical, however, they appear to be useful in improving the level of expression.

The construct can also include about 10%, 20%, 30%, or more of the N-terminal coding region of a gene preferentially expressed in mammary epithelial cells. For example, the N-terminal coding region can correspond to the promoter used, e.g., a goat β-casein N-terminal coding region.

The construct can be prepared using methods known in the art. The construct can be prepared as part of a larger plasmid. Such preparation allows the cloning and selection of the correct constructions in an efficient manner. The construct can be located between convenient restriction sites on the plasmid so that they can be easily isolated from the remaining plasmid sequences for incorporation into the desired mammal.

Insulator Sequences

The DNA constructs used to make a transgenic animal can include at least one insulator sequence. The terms “insulator”, “insulator sequence” and “insulator element” are used interchangeably herein. An insulator element is a control element which insulates the transcription of genes placed within its range of action but which does not perturb gene expression, either negatively or positively. Preferably, an insulator sequence is inserted on either side of the DNA sequence to be transcribed. For example, the insulator can be positioned about 200 bp to about 1 kb, 5′ from the promoter, and at least about 1 kb to 5 kb from the promoter, at the 3′ end of the gene of interest. The distance of the insulator sequence from the promoter and the 3′ end of the gene of interest can be determined by those skilled in the art, depending on the relative sizes of the gene of interest, the promoter and the enhancer used in the construct. In addition, more than one insulator sequence can be positioned 5′ from the promoter or at the 3′ end of the transgene. For example, two or more insulator sequences can be positioned 5′ from the promoter. The insulator or insulators at the 3′ end of the transgene can be positioned at the 3′ end of the gene of interest, or at the 3′end of a 3′ regulatory sequence, e.g., a 3′ untranslated region (UTR) or a 3′ flanking sequence.

A preferred insulator is a DNA segment which encompasses the 5′ end of the chicken β-globin locus and corresponds to the chicken 5′ constitutive hypersensitive site as described in PCT Publication 94/23046, the contents of which is incorporated herein by reference.

Expression of Proteins in the Mammary Gland

It is desirable to express a heterologous protein, e.g., an antibody, in a specific tissue or fluid, e.g., the milk, of a transgenic animal. The heterologous protein can be recovered from the tissue or fluid in which it is expressed. For example, the heterologous proteins (e.g. antibodies) of the present invention can be expressed in the milk of a transgenic animal. Methods for producing a heterologous protein under the control of a mammary gland specific promoter are described below.

Mammary Gland Specific Promoters and Signal Sequences

Useful transcriptional promoters are those promoters that are preferentially activated in mammary epithelial cells, including promoters that control the genes encoding milk proteins such as caseins, beta lactoglobulin (Clark et al., (1989) BIO/TECHNOLOGY 7: 487-492), whey acid protein (Gordon et al. (1987) BIO/TECHNOLOGY 5: 1183-1187), and lactalbumin (Soulier et al., (1992) FEBS Letts. 297: 13). Casein promoters may be derived from the alpha, beta, gamma or kappa casein genes of any mammalian species; a preferred promoter is derived from the goat beta casein gene (DiTullio, (1992) BIO/TECHNOLOGY 10:74-77). The promoter can also be from lactoferrin or butyrophin. Mammary gland specific protein promoter or the promoters that are specifically activated in mammary tissue can be derived from cDNA or genomic sequences. Preferably, they are genomic in origin.

DNA sequence information is available for the mammary gland specific genes listed above, in at least one, and often in several organisms. See, e.g., Richards et al., J. BIOL. CHEM. 256, 526-532 (1981) (α-lactalbumin rat); Campbell et al., NUCLEIC ACIDS RES. 12, 8685-8697 (1984) (rat WAP); Jones et al., J. BIOL. CHEM. 260, 7042-7050 (1985) (rat β-casein); Yu-Lee & Rosen, J. BIOL. CHEM. 258, 10794-10804 (1983) (rat γ-casein); Hall, BIOCHEM. J. 242, 735-742 (1987) (α-lactalbumin human); Stewart, NUCLEIC ACIDS RES. 12, 389 (1984) (bovine αs1 and κ casein cDNAs); Gorodetsky et al., GENE 66, 87-96 (1988) (bovine β casein); Alexander et al., EUR. J. BIOCHEM. 178, 395-401 (1988) (bovine κ casein); Brignon et al., FEBS LETT. 188, 48-55 (1977) (bovine αS2 casein); Jamieson et al., GENE 61, 85-90 (1987), Ivanov et al., BIOL. CHEM. Hoppe-Seyler 369, 425-429 (1988), Alexander et al., NUCLEIC ACIDS RES. 17, 6739 (1989) (bovine β lactoglobulin); Vilotte et al., BIOCHIMIE 69, 609-620 (1987) (bovine α-lactalbumin). The structure and function of the various milk protein genes are reviewed by Mercier & Vilotte, J. DAIRY SCI. 76, 3079-3098 (1993) (incorporated by reference in its entirety for all purposes). If additional flanking sequences are useful in optimizing expression of the heterologous protein, such sequences can be cloned using the existing sequences as probes. Mammary-gland specific regulatory sequences from different organisms can be obtained by screening libraries from such organisms using known cognate nucleotide sequences, or antibodies to cognate proteins as probes.

Useful signal sequences are milk-specific signal sequences or other signal sequences which result in the secretion of eukaryotic or prokaryotic proteins. Preferably, the signal sequence is selected from milk-specific signal sequences, i.e., it is from a gene which encodes a product secreted into milk. Preferably, the milk-specific signal sequence is related to the mammary gland specific promoter used in the construct, which are described below. The size of the signal sequence is not critical. All that is required is that the sequence be of a sufficient size to effect secretion of the desired recombinant protein, e.g., in the mammary tissue. For example, signal sequences from genes coding for caseins, e.g., alpha, beta, gamma or kappa caseins, beta lactoglobulin, whey acid protein, and lactalbumin can be used.

A cassette which encodes a heterologous antibody, e.g., a modified IgG4 antibody, can be assembled as a construct. For example, the construct can include a promoter for a specific tissue, e.g., for mammary epithelial cells, e.g., a casein promoter, a milk-specific signal sequence, e.g., a casein signal sequence, e.g., and a DNA encoding the heterologous antibody, e.g., a modified IgG4 antibody. A construct can be prepared using methods known in the art. The construct can be prepared as part of a larger plasmid. Such preparation allows the cloning and selection of the correct constructions in an efficient manner. The construct can be located between convenient restriction sites on the plasmid so that they can be easily isolated from the remaining plasmid sequences for incorporation into the desired mammal.

Oocytes

Oocytes can be obtained at various times during an animal's reproductive cycle. Oocytes at various stages of the cell cycle can be obtained and then induced in vitro to enter a particular stage of meiosis. For example, oocytes cultured on serum-starved medium become arrested in metaphase. In addition, arrested oocytes can be induced to enter telophase by serum activation.

Oocytes can be matured in vitro before they are used to form a reconstructed embryo. This process usually requires collecting immature oocytes from mammalian ovaries, e.g., a caprine ovary, and maturing the oocyte in a medium prior to enucleation until the oocyte reaches the desired meiotic stage, e.g., metaphase or telophase. In addition, oocytes that have been matured in vivo can be used to form a reconstructed embryo.

Oocytes can be collected from a female mammal during superovulation. Briefly, oocytes, e.g., caprine oocytes, can be recovered surgically by flushing the oocytes from the oviduct of the female donor. Methods of inducing superovulation in goats and the collection of caprine oocytes is described herein.

Transfer of Reconstructed Embryos

A reconstructed embryo can be transferred to a recipient and allowed to develop into a cloned or transgenic mammal. For example, the reconstructed embryo can be transferred via the fimbria into the oviductal lumen of each recipient. In addition, methods of transferring an embryo to a recipient mammal are known in the art and described, for example, in Ebert et al. (1994) Bio/Technology 12:699.

Purification of Proteins from Milk

A preparation, as used herein, refers to two or more antibody molecules. The preparation can be produced by one or more than one transgenic animal. It can include molecules of differing glycosylation or it can be homogenous in this regard.

A “purified preparation”, “substantially pure preparation of antibodies”, or “isolated antibodies as used herein, refers to an antibody that is substantially free of material with which it occurs in the milk of a transgenic mammal. The antibody is also preferably separated from substances, e.g., gel matrix, e.g., polyacrylamide, which is used to purify it. In one embodiment, the language “substantially free” includes preparations of an antibody having less than about 30% (by dry weight) of non-antibody material (also referred to herein as a “milk impurity” or “milk component”), more preferably less than about 20% of non-antibody material, still more preferably less than about 10% of non-antibody material, and most preferably less than about 5% non-antibody material. Non-antibody material includes casein, lipids (e.g., soluble lipids and phospholipids), lactose and other small molecules (e.g., glucose, galactose), small peptides (e.g., microbial peptides and anti-microbial peptides) and other milk proteins (e.g., whey proteins such as β-lactoglobulin and α-lactalbumin, lactoferrin, and serum albumin). The antibodies preferably constitute at least 10, 20, 50 70, 80 or 95% dry weight of the purified preparation. Preferably, the preparation contains: at least 1, 10, or 100 μg of the antibodies; at least 1, 10, or 100 mg of the antibodies. In addition, the purified preparation preferably contains about 70%, 75%, 80%, 85%, 90%, 95%, 98% assembled antibodies.

Antibodies (and fragments thereof) can be isolated from milk using standard protein purification methods known in the art. For example, the methods of Kutzko et al. (U.S. Pat. No. 6,268,487) can be utilized to purify antibodies and/or fragments of the present invention.

Milk proteins are often isolated by a combination of processes. For example, raw milk can first be fractionated to remove fats, for example, by skimming, centrifugation, sedimentation (H. E. Swaisgood, Developments in Dairy Chemistry, in: CHEMISTRY OF MILK PROTEIN, Applied Science Publishers, NY, 1982), acid precipitation (U.S. Pat. No. 4,644,056) or enzymatic coagulation with rennin or chymotrypsin (Swaisgood, ibid.). Next, the major milk proteins may be fractionated into either a clear solution or a bulk precipitate from which the specific protein of interest may be readily purified. As another example, French Patent No.# 2,487,642 describes the isolation of milk proteins from skim milk or whey by membrane ultrafiltration in combination with exclusion chromatography or ion exchange chromatography. Whey is first produced by removing the casein by coagulation with rennet or lactic acid. U.S. Pat. No. 4,485,040 describes the isolation of an alpha-lactoglobulin-enriched product in the retentate from whey by two sequential ultrafiltration steps. U.S. Pat. No. 4,644,056 provides a method for purifying immunoglobulin from milk or colostrum by acid precipitation at pH 4.0-5.5, and sequential cross-flow filtration first on a membrane with 0.1-1.2 micrometer pore size to clarify the product pool and then on a membrane with a separation limit of 5-80 kd to concentrate it. U.S. Pat. No. 4,897,465 teaches the concentration and enrichment of a protein such as immunoglobulin from blood serum, egg yolks or whey by sequential ultrafiltration on metallic oxide membranes with a pH shift. Filtration is carried out first at a pH below the isoelectric point (pI) of the selected protein to remove bulk contaminants from the protein retentate, and next at a pH above the pI of the selected protein to retain impurities and pass the selected protein to the permeate. A different filtration concentration method is taught by European Patent No. EP 467 482 B1 in which defatted skim milk is reduced to pH 3-4, below the pI of the milk proteins, to solubilize both casein and whey proteins. Three successive rounds of ultrafiltration or diafiltration then concentrate the proteins to form a retentate containing 15-20% solids of which 90% is protein.

As another example, milk can initially be clarified. A typical clarification protocol can include the following steps:
(a) diluting milk 2:1 with 2.0 M Arginine-HCl pH 5.5;
(b) spinning diluted sample in centrifuge for approximately 20 minutes at 4-8° C.;
(c) cooling samples for approximately 5 minutes on ice to allow fat sitting on top to solidify;
(d) removing fat pad by “popping” it off the top with a pipette tip; and
(e) decanting of supernatant into a clean tube.

Further purification of proteins can be achieved using any method for protein purification known in the art, e.g. by methods as described above.

### EXAMPLES

### Example 1

### Modification of Antibodies

An antibody heavy chain can be modified using oligonucleotide mutagenesis. Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. USA, 75: 5765[1978]).

To effectuate a change from serine to proline at amino acid number 241 of the hinge region, oligonucleotide mutagenesis can be employed using the oligo S241P that will change the serine to proline. The resulting mutant form can be used to generate transgenic mice. The transgenic mice can be milked, and the milk tested for the presence of the antibody and the relative amount of the “half molecule.” The sequence of a hinge region of an IgG4 antibody and the oligonucleotideS241P which can be used to mutagenize it are as follows:

```
IGG4 HINGE REGION
1668 TCTGGA GAG TCG AAA TAT GGT CCC CCA TGC CCA TCA TGC CCA
GGTAAGCCAACCCAGGCCT
                1^{R}_{S} Glu Ser Lys Tyr Gly Pro Pro Cys Pro Ser Cys Pro
S241P OLIGO
      GGT CCC CCA TGT CCT CCC TGC CCA GGT AAG CCA
^{R}_{S} Gly Pro Pro Cys Pro Pro Cys Pro Gly Lys Pro
```

Further, the entire hinge region of an IgG antibody can be replaced with the hinge region of another antibody. To effectuate this change, an oligonucleotide that codes for the an exon containing the replacement hinge region can be used. The sequence of a hinge region of an IgG4 antibody and an oligonucleotide which contains an IgG2 replacement hinge region are as follows:

```
IGG4 HINGE REGION
1662 CTTCTCTCTGCA GAG TCC AAA TAT GGT CCC CCA TGC CCA TCA TGC CCA GGTCCGCCAACCCAGGC
                              1^{R}_{S} Glu Ser Lys Tyr Gly Pro Pro Cys Pro Ser Cys Pro
IGG2 HINGE REGION
1729 CTTCTCTCTGCA GAG CGC AAA TGT TGT GTC GAG TGC CCA CCG TGC CCA GGTCCGCCAACCCAGGC
                                1^{R}_{S} Glu Arg Lys Cys Cys Val Glu Cys Pro Pro Cys Pro
```

The N-linked glycosylation site on the CH2 of an IgG heavy chain can be eliminated via oligonucleotide mutagenesis using an oligo that causes a change from asparagine to glutamine in the consensus site. The sequence of an oligonucleotide that can effectuate such a change is as follows:

2014 GAG GAG CAG TTC CAG TCT ACT TAC CGA GTG GTC
1^{R}_{S} Glu Glu Gln Phe Gln Ser Thr Tyr Arg Val Val

Testing of Mutagenized Versions of Antibodies

The light chain and mutagenized heavy chain are ligated to the casein promoter and used to generate transgenic mice. Mice are then tested for expression of the antibody as well as the half antibody.

Transgenic Animals

A founder (F_{O}) transgenic goat can be made by transfer of fertilized goat eggs that have been microinjected with a construct. The methodologies that follow in this section can be used to generate transgenic goats. The skilled practitioner will appreciate that such procedures can be modified for use with other animals.

Goat Species and Breeds:

Swiss origin goats, e.g., the Alpine, Saanen, and Toggenburg breeds, are useful in the production of transgenic goats.

The sections outlined below briefly describe the steps required in the production of transgenic goats. These steps include superovulation of female goats, mating to fertile males and collection of fertilized embryos. Once collected, pronuclei of one-cell fertilized embryos are microinjected with DNA constructs. All embryos from one donor female are kept together and transferred to a single recipient female if possible.

Goat Superovulation:

The timing of estrus in the donors is synchronized on Day 0 by 6 mg subcutaneous norgestomet ear implants (Syncromate-B, CEVA Laboratories, Inc., Overland Park, Kans.). Prostaglandin is administered after the first seven to nine days to shut down the endogenous synthesis of progesterone. Starting on Day 13 after insertion of the implant, a total of 18 mg of follicle-stimulating hormone (FSH-Schering Corp., Kenilworth, N.J.) is given intramuscularly over three days in twice-daily injections. The implant is removed on Day 14. Twenty-four hours following implant removal the donor animals are mated several times to fertile males over a two-day period (Selgrath, et al., Theriogenology, 1990. pp. 1195-1205).

Embryo Collection:

Surgery for embryo collection occurs on the second day following breeding (or 72 hours following implant removal). Superovulated does are removed from food and water 36 hours prior to surgery. Does are administered 0.8 mg/kg Diazepam (Valium®), IV, followed immediately by 5.0 mg/kg Ketamine (Keteset), IV. Halothane (2.5%) is administered during surgery in 2 L/min oxygen via an endotracheal tube. The reproductive tract is exteriorized through a midline laparotomy incision. Corpora lutea, unruptured follicles greater than 6 mm in diameter, and ovarian cysts are counted to evaluate superovulation results and to predict the number of embryos that should be collected by oviductal flushing. A cannula is placed in the ostium of the oviduct and held in place with a single temporary ligature of 3.0 Prolene. A 20 gauge needle is placed in the uterus approximately 0.5 cm from the uterotubal junction. Ten to twenty ml of sterile phosphate buffered saline (PBS) is flushed through the cannulated oviduct and collected in a Petri dish. This procedure is repeated on the opposite side and then the reproductive tract is replaced in the abdomen. Before closure, 10-20 ml of a sterile saline glycerol solution is poured into the abdominal cavity to prevent adhesions. The linea alba is closed with simple interrupted sutures of 2.0 Polydioxanone or Supramid and the skin closed with sterile wound clips.

Fertilized goat eggs are collected from the PBS oviductal flushings on a stereomicroscope, and are then washed in Ham's F12 medium (Sigma, St. Louis, Mo.) containing 10% fetal bovine serum (FBS) purchased from Sigma. In cases where the pronuclei are visible, the embryos is immediately microinjected. If pronuclei are not visible, the embryos are placed in Ham's F12 containing 10% FBS for short term culture at 37° C. in a humidified gas chamber containing 5% CO₂ in air until the pronuclei become visible (Selgrath, et al., Theriogenology, 1990. pp. 1195-1205).

Microinjection Procedure:

One-cell goat embryos are placed in a microdrop of medium under oil on a glass depression slide. Fertilized eggs having two visible pronuclei are immobilized on a flame-polished holding micropipet on a Zeiss upright microscope with a fixed stage using Normarski optics. A pronucleus is microinjected with the DNA construct of interest, e.g., a BC355 vector containing a coding sequence of interest operably linked to the regulatory elements of the goat beta-casein gene, in injection buffer (Tris-EDTA) using a fine glass microneedle (Selgrath, et al., Theriogenology, 1990. pp. 1195-1205).

Embryo Development:

After microinjection, the surviving embryos are placed in a culture of Ham's F12 containing 10% FBS and then incubated in a humidified gas chamber containing 5% CO₂ in air at 37° C. until the recipient animals are prepared for embryo transfer (Selgrath, et al., THERIOGENOLOGY, 1990. p. 1195-1205).

Preparation of Recipients:

Estrus synchronization in recipient animals is induced by 6 mg norgestomet ear implants (Syncromate-B). On Day 13 after insertion of the implant, the animals are given a single non-superovulatory injection (400 I.U.) of pregnant mares serum gonadotropin (PMSG) obtained from Sigma. Recipient females are mated to vasectomized males to ensure estrus synchrony (Selgrath, et al., THERIOGENOLOGY, 1990. pp. 1195-1205).

Embryo Transfer:

All embryos from one donor female are kept together and transferred to a single recipient when possible. The surgical procedure is identical to that outlined for embryo collection outlined above, except that the oviduct is not cannulated, and the embryos are transferred in a minimal volume of Ham's F12 containing 10% FBS into the oviductal lumen via the fimbria using a glass micropipet. Animals having more than six to eight ovulation points on the ovary are deemed unsuitable as recipients. Incision closure and post-operative care are the same as for donor animals (see, e.g., Selgrath, et al., Theriogenology, 1990. pp. 1195-1205).

Monitoring of Pregnancy and Parturition:

Pregnancy is determined by ultrasonography 45 days after the first day of standing estrus. At Day 110 a second ultrasound exam is conducted to confirm pregnancy and assess fetal stress. At Day 130 the pregnant recipient doe is vaccinated with tetanus toxoid and Clostridium C&D. Selenium and vitamin E (Bo-Se) are given IM and Ivermectin was given SC. The does are moved to a clean stall on Day 145 and allowed to acclimatize to this environment prior to inducing labor on about Day 147. Parturition is induced at Day 147 with 40 mg of PGF2a (Lutalyse®, Upjohn Company, Kalamazoo Mich.). This injection is given IM in two doses, one 20 mg dose followed by a 20 mg dose four hours later. The doe is under periodic observation during the day and evening following the first injection of Lutalyse® on Day 147. Observations are increased to every 30 minutes beginning on the morning of the second day. Parturition occurred between 30 and 40 hours after the first injection. Following delivery the doe is milked to collect the colostrum and passage of the placenta is confirmed.

Verification of the Transgenic Nature of F₀ Animals:

To screen for transgenic F₀ animals, genomic DNA is isolated from two different cell lines to avoid missing any mosaic transgenics. A mosaic animal is defined as any goat that does not have at least one copy of the transgene in every cell. Therefore, an ear tissue sample (mesoderm) and blood sample are taken from a two day old F₀ animal for the isolation of genomic DNA (Lacy, et al., A LABORATORY MANUAL, 1986, Cold Springs Harbor, N.Y.; and Herrmann and Frischauf, METHODS ENZYMOLOGY, 1987. 152: pp. 180-183). The DNA samples are analyzed by the polymerase chain reaction (Gould, et al., Proc. Natl. Acad. Sci, 1989. 86:pp. 1934-1938) using primers SPECIFIC FOR HUMAN DECORIN GENE AND BY SOUTHERN BLOT ANALYSIS (THOMAS, PROC Natl. Acad. Sci., 1980. 77:5201-5205) using a random primed human decorin cDNA probe (Feinberg and Vogelstein, Anal. Bioc., 1983. 132: pp. 6-13). Assay sensitivity is estimated to be the detection of one copy of the transgene in 10% of the somatic cells.

Generation and Selection of Production Herd

The procedures described above can be used for production of transgenic founder (F₀) goats, as well as other transgenic goats. The transgenic F₀ founder goats, for example, are bred to produce milk, if female, or to produce a transgenic female offspring if it is a male founder. This transgenic founder male, can be bred to non-transgenic females, to produce transgenic female offspring.

Transmission of Transgene and Pertinent Characteristics

Transmission of the transgene of interest, in the goat line is analyzed in ear tissue and blood by PCR and Southern blot analysis. For example, Southern blot analysis of the founder male and the three transgenic offspring shows no rearrangement or change in the copy number between generations. The Southern blots are probed with human decorin cDNA probe. The blots are analyzed on a Betascope 603 and copy number determined by comparison of the transgene to the goat beta casein endogenous gene.

Evaluation of Expression Levels

The expression level of the transgenic protein, in the milk of transgenic animals, is determined using enzymatic assays or Western blots.

### Example 2

### Mouse Model of Antibody Hinge Region Change

To check the feasibility of production of recombinant therapeutic antibodies in transgenic animals, the cDNA for the antibody KMK917 was expressed in the mammary gland of transgenic mice. KMK917 was then purified from mouse milk and compared to KMK917 derived from fed batch fermentation of KMK917-transfected Sp2/0 cells. KMK917-transgenic mice were generated at GTC Biotherapeutics, Inc., in Framingham, Mass., The subsequent purification and analytical characterization were performed by a sub-contractor.

Generation of KMK917 Transgenic Mice

The KMK917 coding constructs were generated:
1. 1099/2010 coding for KMK917 wild type
2. 2012/2017 coding for KMK917 hinge mutant (229 Ser→Pro)
3. 2012/2017 coding for KMK917 hinge+Ch2 mutant (229 Ser→Pro,

The mutant constructs were generated with the purpose to reduce the portion of half antibodies observeed in KMK917 material derived from the wild type construct. Based on these constructs a total of 15 transgenic mouse lines were generated (for an overview and labeling of the lines see Table 1a-c). Table 1 contains and estimation of the expression level of KMK917 in the mouse lines made by Western Blotting.

TABLE 1a
Transgenic mouse lines generated with construct 1099/2010 wild type

| | | | | | | | Estim. expr. |
|---|---|---|---|---|---|---|---|
| Mouse line | Generation milked | | | Day of | Approx. | μL PBS | Level |
| (sex) | F0 | F1 | F2 | milking | volume (μL) | added | (mg/mL) |
| 1-73 F | 1-73 | | | 7 | 175 | 700 | <1 |
| | | | | 9 | 225 | 900 | |
| | | | | 13 | 100 | 400 | |
| | | | | Total | 500 μl | 2000 μl | |
| 1-78 M | | 2-119 | | 10 | 150 | 600 | 10+ |
| | | | 3-150 | 8 | 125 | 500 | |
| | | | | 10 | 250 | 1000 | |
| | | | | 14 | 100 | 400 | |
| | | | | Total | 625 μl | 2500 μl | |
| 1-46 M | | 2-138 | | 10 | 50 ul | 200 ul | 10+ |
| | | | 3-145 | Feb. 5, 2002 | 150 | 600 | |
| | | | | Feb. 11, 2002 | 50 | 200 | |
| | | | | Total | 250 μl | 1000 μl | |

TABLE 1b
Transgenic mouse lines generated with construct 2012/2014 hinge
mutant

| Mouse | | | | | Approx. | | Estim. expr. |
|---|---|---|---|---|---|---|---|
| line | Generation milked | | | Day of | volume | μL PBS | Level |
| (sex) | F0 | F1 | F2 | milking | (μl) | added | (mg/ml) |
| 1-4 F | 1-4 | | | 7 | 125 | 500 | 7-10 |
| | | | | 11 | 125 | 500 | |
| | | 2-120 | | 7 | 250 | 1000 | |
| | | | | 11 | 150 | 600 | |
| | | | | 13 | 100 | 400 | |
| | | | | Total | 750 μl | 3000 μl | |
| 1-57 F | 1-57 | | | 10 | 200 | 800 | 4-5 |
| | | | | 13 | 25 | 100 | |
| | | | | 15 | 50 | 200 | |
| | | 2-141 | | 6 | 150 | 600 | |
| | | | | 11 | 100 | 400 | |
| | | 2-143 | | 9 | 200 | 800 | |
| | | | | 9 | 200 | 800 | |
| | | 2-144 | | 7 | 150 | 600 | |
| | | | | 10 | 250 | 1000 | |
| | | | | 12 | 250 | 1000 | |
| | | | | Total | 1575 μl | 6300 μl | |
| 1-62 F | | 2-145 | | 6 | 100 | 400 | 7-10 |
| | | | | 10 | 125 | 500 | (1-62 F) |
| | | | | 12 | 125 | 500 | |
| | | 2-147 | | 6 | 75 | 300 | |
| | | | | Total | 425 μl | 1700 μl | |
| 1-65 M | | 2-149 | | 7 | 50 | 200 | |
| | | | | 10 | 50 | 200 | |
| | | 2-150 | | 7 | 150 | 600 | |
| | | | | 10 | 100 | 400 | |
| | | | | 12 | 200 | 800 | |
| | | | | Total | 550 μl | 2200 μl | |
| 1-76 F | 1-76 | | | 6 | 150 | 600 | |
| | | | | 9 | 250 | 1000 | |
| | | | | 9 | 250 | 1000 | |
| | | | | 11 | 200 | 800 | |
| | | | | 11 | 200 | 800 | |
| | | | | Total | 1050 μl | 4200 μl | |
| 1-96 F | 1-96 | | | 6 | 50 | 200 | |
| | | | | 9 | 250 | 1000 | |
| | | | | 11 | 200 | 800 | |
| | | | | Total | 500 μl | 2000 μl | |

TABLE 1c
Transgenic mouse lines generated with construct 2012/2017

| Mouse | | | | | Approx. | | |
|---|---|---|---|---|---|---|---|
| line | Generation milked | | | Day of | volume | μL PBS | Estim. expr. |
| (sex) | F0 | F1 | F2 | milking | (μl) | added | Level (mg/ml) |
| 1-7 M | | 2-92 | | 9 | 200 | 800 | |
| | | | | 11 | 100 | 400 | |
| | | 2-93 | | 6 | 100 | 400 | |
| | | | | 8 | 75 | 300 | |
| | | 2-94 | | 5 | 125 | 500 | |
| | | | | 7 | 150 | 600 | |
| | | | | 9 | 75 | 300 | |
| | | | | Total | 825 ul | 3300 ul | |
| 1-13 F | | 2-87 | | 5 | 175 | 700 | ˜1 |
| | | | | 7 | 200 | 800 | (1-13 F) |
| | | | | 11 | 125 | 500 | |
| | | | | Total | 500 ul | 2000 ul | |
| 1-25 F | | 2-108 | | 6 | 50 | 200 | ˜1.5 |
| | | | | 8 | 100 | 400 | (1-25 F) |
| | | | | 10 | 75 | 300 | |
| | | 2-109 | | 6 | 150 | 600 | |
| | | | | 8 | 50 | 200 | |
| | | | | 12 | 125 | 500 | |
| | | | | Total | 550 ul | 2200 ul | |
| 1-30 F | | 2-116 | | 6 | 250 | 1000 | ˜1 |
| | | | | 8 | 200 | 800 | (1-30 F) |
| | | | | 12 | 125 | 500 | |
| | | 2-118 | | 5 | 200 | 800 | |
| | | | | 7 | 250 | 1000 | |
| | | | | 11 | 150 | 600 | |
| | | | | 12 | 150 | 600 | |
| | | | | Total | 1325 ul | 5300 ul | |
| 1-36 F | 1-36 | | | 5 | 125 | 500 | 10+ |
| | | | | 9 | 100 | 400 | |
| | | | | 11 | 125 | 500 | |
| | | 2-126 | | 5 | 50 | 200 | |
| | | 2-127 | | 7 | 100 | 400 | |
| | | | | Total | 500 ul | 2000 ul | |
| 1-61 M | | 2-129 | | 8 | 200 | 800 | |
| | | | | 8 | 200 | 800 | |
| | | | | 12 | 150 | 600 | |
| | | | | 12 | 150 | 600 | |
| | | 2-131 | | 6 | 125 | 500 | |
| | | | | 6 | 125 | 500 | |
| | | | | 10 | 250 | 1000 | |
| | | | | 12 | 200 | 800 | |
| | | 2-133 | | 6 | 175 | 700 | |
| | | | | 6 | 175 | 700 | |
| | | | | 8 | 250 | 1000 | |
| | | | | 10 | 150 | 600 | |
| | | | | Total | 2150 ul | 8600 ul | |

Purification and Characterization of KMK917 Derived from the Milk of Transgenic Mice

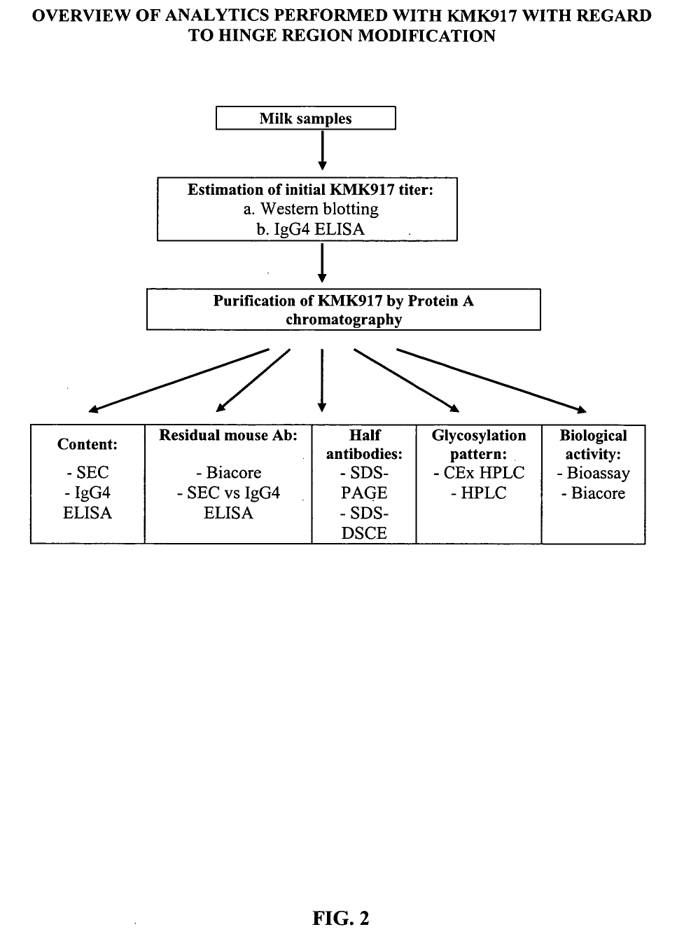

Milk samples from a total of 15 transgenic mouse lines were harvested (F0, F1, and/or F2 generation) and diluted with PBS (for details see Table 1). Samples were then purified and characterized for the KMK917 antibody. For an overview on the analytics performed see FIG. 2.

For removal of the colloidal milk components, the pre-diluted milk samples were centrifuged at high speed on a Sorval centrifuge for 30 minutes (SS-34 rotor at 20,000 rpm), the supernatant was sucked off from the pellet and the upper fat-layer removed by means of a syringe. The slightly opalescent supernatant was filtered through a 0.22 um Millex-GV filter and loaded on a 1 ml Protein A column (MabSelect, APB). The bound antibody was eluted with 20 mM sodium citrate/citric acid pH 3.2. The antibody fraction was adjusted to pH 5.5, sterile filtered and stored at 4° C.

Determination of KMK917 Content in the Milk of Transgenic Mice

Using a commercially available ELISA kit for the detection of human IgG4, the concentration of KMK917 was measured in the pre-diluted mouse milk samples. The values corresponding to the content of KMK917 in undiluted mouse milk are given in Table 2.

TABLE 2
Concentration of KMK917 in the milk of transgenic lines

| | | | | Content in | | |
|---|---|---|---|---|---|---|
| | | | | purified | | |
| | | | | fractions | | Amount of |
| | | Content in milk (mg/mL) | | (mg/mL) | | KMK917 |
| Con- | | | Back calculated | | IgG4 | (mg) |
| struct | Line | IgG4 ELISA | from SEC | SEC | ELISA | SEC |

1099/2010 wild type

| | | | | | |
|---|---|---|---|---|---|
| 1-73 | 3.2 | — | — | — | — |
| 1-78 | >10 | 22.1 | 3.2 | 3.4 | 9.1 |
| 1-46 | 8.5 | 7.7 | 0.8 | 1.0 | 1.7 |

2012/2014 hinge mutant

| | | | | | |
|---|---|---|---|---|---|
| 1-4 | — | 4.5 | 0.9 | 1.1 | 1.8 |
| 1-57 | 3.5 | — | — | — | — |
| 1-62 | 16 | — | — | — | — |
| 1-65 | 11 | 10.9 | 1.9 | 2.8 | 4.6 |
| 1-76 | 0.8 | — | — | — | — |
| 1-96 | 3.4 | — | — | — | — |

2012/2017 hinge and Ch2 mutant

| | | | | | |
|---|---|---|---|---|---|
| 1-7 | 5.5 | 3.2 | 0.9 | 1.1 | 1.9 |
| 1-13 | 2.3 | — | — | — | — |
| 1-25 | 1.5 | 4.7 | 0.8 | 0.9 | 1.4 |
| 1-30 | 4.5 | — | — | — | — |
| 1-36 | >10 | 9.7 | 1.4 | 1.5 | 3.3 |
| 1-61 | 1 | — | — | — | — |

Subsequently, KMK917 from selected mouse lines (2 or 3 of each construct) was purified by Protein A chromatography as described in 3.2. Size-exclusion HPLC (SEC) was then used to determine the content of KMK917 in the antibody fractions (Table 2). The total amount of KMK917 available for further analyses is also shown in Table 2.

SEC analysis showed that all antibody samples contained monomeric antibody to more than 95%. Based upon the measured KMK917 content in the antibody fractions and the volume used for Protein A purification, the content of KMK917 in the mouse milk samples was back calculated. Back calculated concentrations of KMK917 in mouse milk were found to be between 3.2 and 22.1 mg/mL correlating very well with the values measured directly in mouse milk by IgG4 ELISA (Table 2).

Presence of Mouse Antibodies in Purified KMK917 Material

Since purification using Protein A enriches not only human IgG isotypes but also some isoforms of mouse antibodies which may be present in milk, purified KMK917 was checked for the presence of mouse immunoglobulins. Using the SPR technology (Biacore 3000) and immobilized anti-mouse IgG as a “capture molecule” no or only very low amounts of murine IgG subclasses were detected in the purified KMK917 material (≦0.1%). This finding is supported by the fact that concentration measurements of purified material by both SEC and a human IgG4 ELISA revealed very comparable results (Table 2). A significant amount of mouse immunoglobulins would have been indicated by higher concentration level determined by SEC since this method measures not only KMK917 but also mouse antibodies. In contrast, the ELISA is specific for human IgG4 and therefore detects only KMK917.

Presence and Amount of “Half Antibodies”

The amount of half antibodies present in purified KMK917 material from the transgenic mouse lines was determined using SDS-PAGE and SDS-DSCE. SDS-PAGE revealed a higher portion of half antibodies in the samples of wild type-transfected mice in comparison to the samples from mice transfected with the mutated construct.

These results were confirmed by SDS-DSCE revealing 24 and 34% half antibodies in the KMK917 material derived from transgenic lines generated with the wild type construct. In KMK917 material from the mutant constructs, the portion of half antibodies was found to be well below 5%, especially in the material derived from the single mutant construct (see summary in Table 4).

To assess the biological activity of KMK917 derived from the different constructs, a fluorescence-based cellular assay was used in which KMK917 competes with a cellular receptor for the binding of its receptor target. Compared to cell culture (Sp2/0)-derived KMK917, full biological activity was found for KMK917 derived from both, wild type and mutant-transfected mice (see Table 4).

For further characterization, the kinetic rate constants for the association and dissociation of KMK917 with its ligand target were determined using the SPR technology (Biacore 3000). In all samples, rate constants of transgenic mice-derived material were found to be comparable to the values found for the Sp2/0-derived KMK917. This indicates that the binding affinity and biological activity of KMK917 is (1) similar if expressed in transgenic mice or in the cell line Sp2/0 and (2) is not influenced by the mutations introduced into the cDNA.

TABLE 4
Summary of analytical characterization of KMK917 derived from transgenic mice

| | | Wild type | | Hinge mutant | | Hinge + Ch2 mutant | | |
|---|---|---|---|---|---|---|---|---|
| | | Line 1-46 | Line 1-78 | Line 1-4 | Line 1-65 | Line 1-7 | Line 1-25 | Line 1-36 |
| Analytical test | | HT560/1 | HT557/4 | HT557/2 | HT560/2 | HT560/3 | HT557/1 | HT557/3 |
| Estimated amount of | Biacore | <0.1 | 0 | <0.1 | <0.1 | <0.1 | ˜0.1 | <0.1 |
| mouse Ab (%) | | | | | | | | |
| Half antibodies (%) | SDS-DSCE | 24.0 | 34.4 | 1.8 | 1.6 | 4.6 | 2.9 | 4.9 |
| | SDS-PAGE | 38.1 | 43.5 | 2.4 | 3.7 | 7.6 | 4.0 | 4.5 |
| Biological activity | FACS | 105 | 99 | 115 | 116 | 109 | 94/98 | 122 |
| (Relative potency in %) | | | | | | | | |
| Biological affinity | Biacore | 5.3 | 4.2 | 4.3 | 4.8 | 4.9 | 4.7 | 4.4 |
| (association and | ka (10⁶ | | | | | | | |
| dissociation rate | (Ms)⁻¹) | | | | | | | |
| constants ka and kd; | Biacore | 3.5 | 3.7 | 3.0 | 3.6 | 4.2 | 2.4 | 3.8 |
| ka (KMK ref) = 4.1 | kd (10⁻⁴s⁻¹) | | | | | | | |
| kd (KMK ref) = 4.7) | | | | | | | | |
| Heterogenicity of | CEx-HPLC | ++ | ++ | +++ | +++ | +++ | nd | +++ |
| elution profile (KMK | | | | | | | | |
| ref = +) | | | | | | | | |

*Estimated by Western Blotting
nd = not determined

Glycosylation Pattern

Cation-exchange HPLC was used to analyze the purified KMK917 material. The specific method used is able to achieve separation of the C-terminal des-Lys variants of antibody (variant K0, variant K1 and variant K2) and also resolution of different glycoforms of the antibody, for instance sialidated from non-sialidated glycoforms but also mannose-type from complex-type glycoforms.

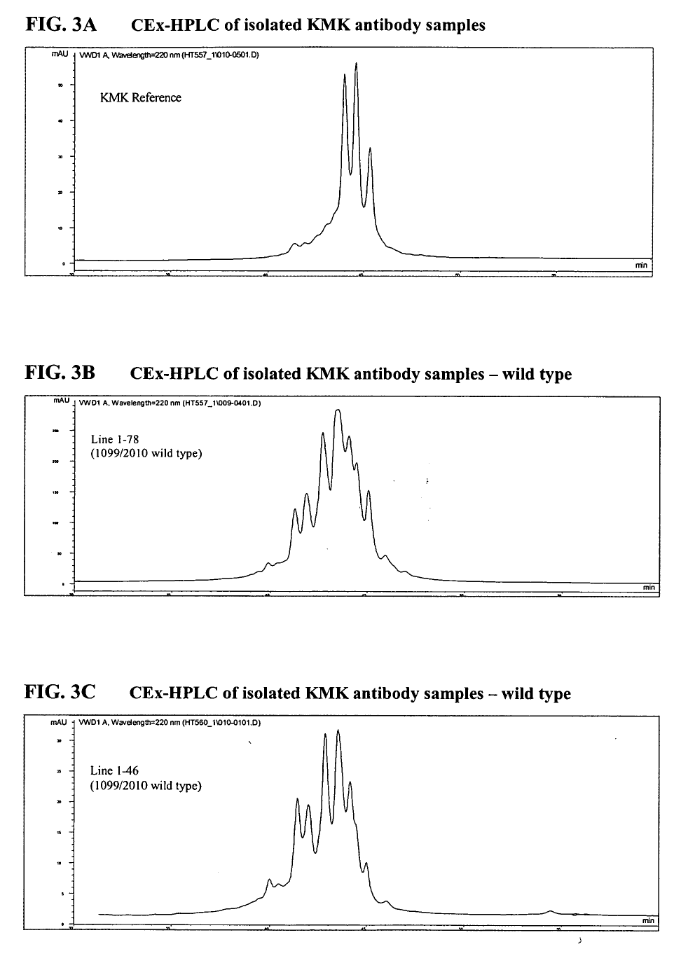

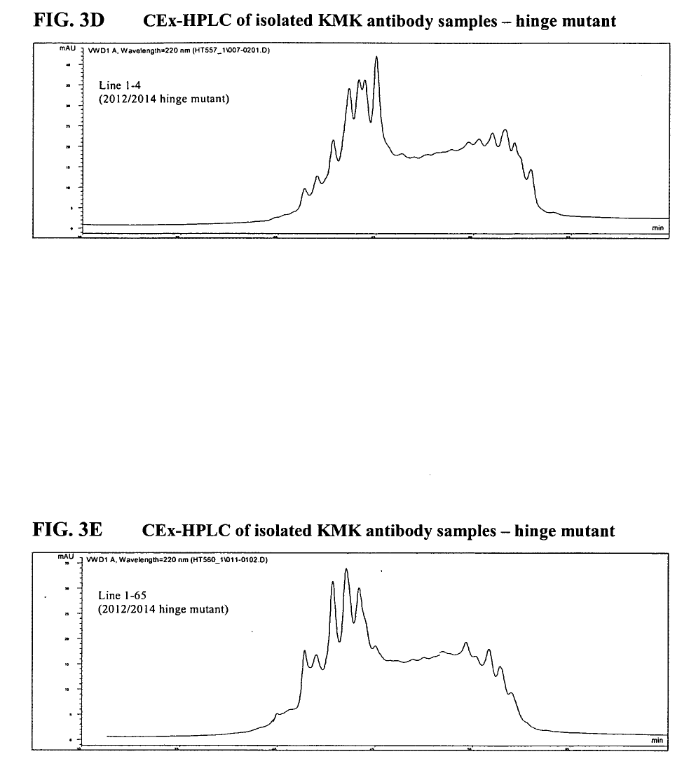

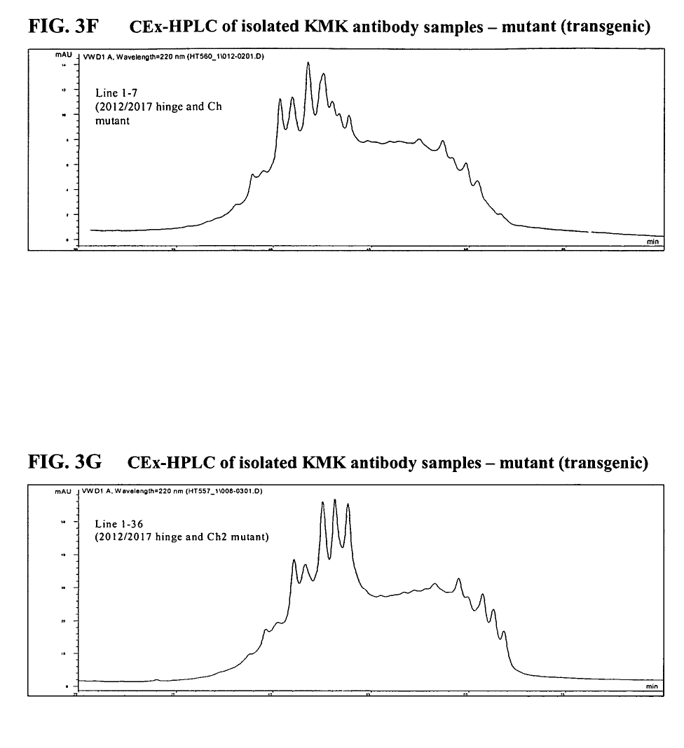

FIGS. 3a-3g show the elution profile of the KMKreference sample obtained from cell culture and the elution profiles of the antibodies obtained from the milk samples. The three main peaks of the reference correspond to the K0, K1 and K2 variants.

The samples obtained from transgenic milk are more heterogeneous. The two wild type samples show additional peaks eluting earlier with respect to reference and could be caused by sialidated glycoforms. The antibody samples obtained from the mutant lines show a very heterogeneous pattern with variants also eluting behind the reference.

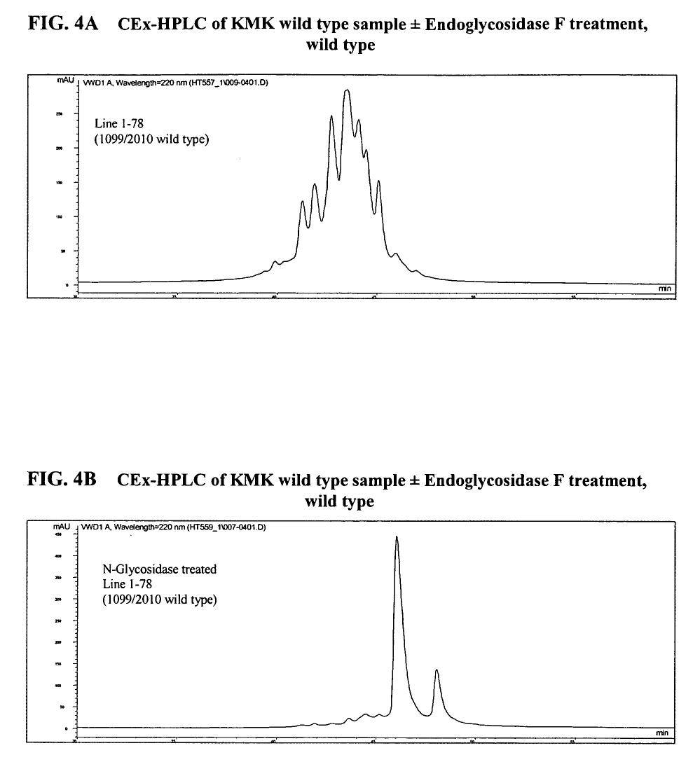

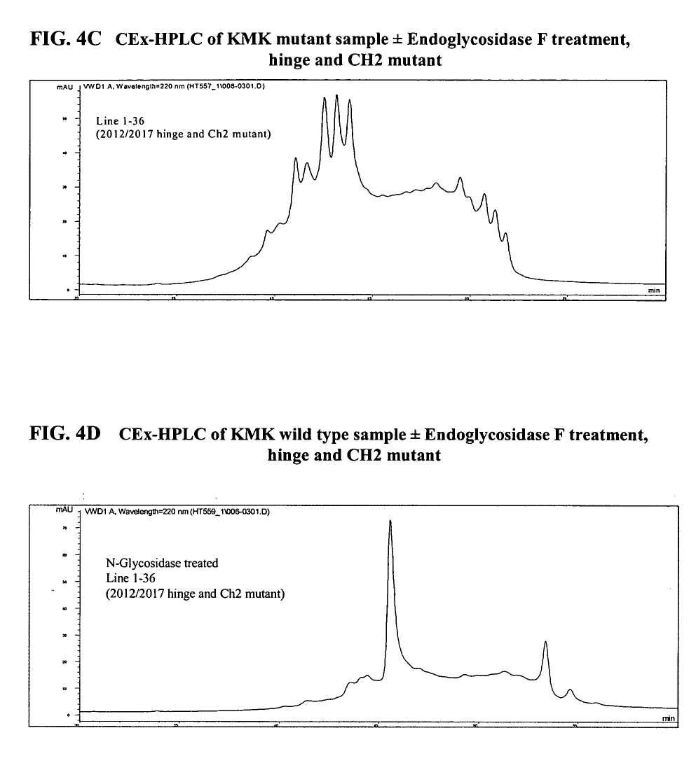

To elucidate how much of the heterogeneity observed is caused by different glycosylation forms, a wild type and mutant sample was deglycosylated by N-Glycosidase treatment. FIGS. 4a-4d show the CEx-HPLC profile of the wild type sample before and after glycosidase treatment. The wild type sample yielded after deglycosylation a much more homogeneous pattern. The two peaks obtained in the ratio 4:1 very likely correspond to the K0 and K1 form of the antibody. From these results it can be concluded that the heterogeneity observed in the wild type antibody is caused mainly by glycoform variants.

The mutant antibody from line 1-36 also yielded two main peaks in about the same ratio. However, the two peaks elute much more distant from each other and were accompanied by a subset of side-peaks (see FIG. 3b). Such a behavior could be interpreted by the presence of different antibody conformers in the mutant variant, potentially caused by partial unfolding. Thus, the broad heterogeneity observed in CEx-HPLC analyses of the mutant antibodies appears to be caused not only by different glycoforms but also by other sources.

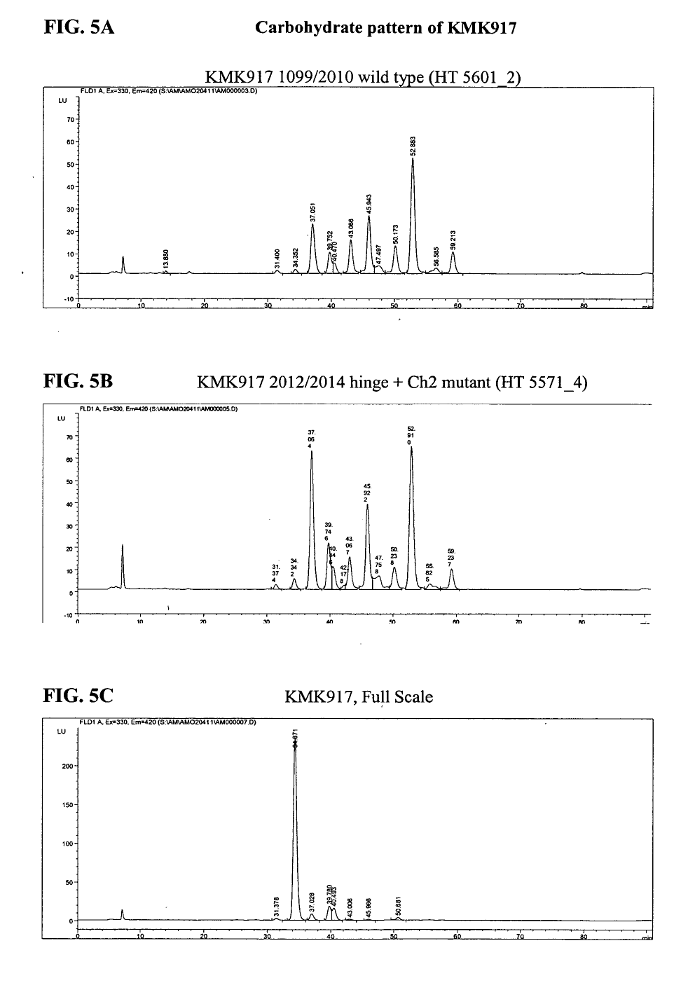

Further structural eludication of the carbohydrate side chain has been performed with purified KMK917. After enzymatic cleavage with PNGase F the carbohydrate side chain was isolated and labeled with 2-aminobenzamide. The individual carbohydrate structures could be separated on HPLC using a Glyco Sep N-column and were quantified by fluorescence detection. FIG. 5a-5c show the chromatograms of analyzed KMK917 from:
a) transgenic mice, wild type
b) transgenic mice, mutant
c) cell culture

The chromatograms show that the carbohydrate pattern of KMK917 from transgenic mice is significantly different compared with the antibody isolated from cell culture. The pattern of the mutant is qualitatively identical with the wild type, and shows only some quantitative differences. When compared with other well known structures of carbohydrate side chains, several peaks could be assigned definitely already from the HPLC pattern. The molecular structures are shown in Table 3.

TABLE 3
Molecular structure of carbohydrate side chains

| Peak # | RT (min) | Carbohydrate structure |
|---|---|---|
| 1 | 31.4 | ? |
| 2 | 34.3 | G 0 |
| 3 | 37.1 | Man 5 |
| 4 + 5 | 39.7 + 40.4 | G 1 |
| 6 | 43.1 | Man 6 |
| 7 | 45.9 | G 2 |
| 8 | 47.5 | ? |
| 9 | 50.2 | ? |
| 10 | 52.9 | ? |
| 11 | Ca. 56 | ? |
| 12 | 59.2 | ? |

To confirm the molecular structures obtained from HPLC and to get some additional information about the late eluting peaks, the carbohydrate mixture has also been analyzed on MALDI-MS. With MALDI-MS in the negative mode one additional structure, the sialinic acid containing carbohydrate, BiG2S1 is proposed.

The expression of KMK917 in the mammary gland of transgenic mice yielded titers of KMK917 in mouse milk between 3.2 and 22.1 mg/mL. Further characterization of KMK917 derived from three different KMK917 construct showed that the amount of “half antibodies” is high (24 and 34%, resp.) in the material derived from the wild type construct 1099/2010. Introduction of the 229 Ser→Pro mutation (constructs 2012/2014 hinge and 2012/2017 hinge+Ch2) significantly reduced the amount of “half antibodies” to values below 2% for 2012/2014 and below 5% for 2012/2017. The biological activity of the material obtained from all three constructs revealed no differences when compared to cell culture-derived KMK917.

It is to be understood that, while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of producing an antibody in the milk of a transgenic mammal, comprising:
providing a transgenic mammal whose somatic and germ cells comprise a sequence encoding an exogenous heavy chain variable region or antigen binding fragment thereof, at least one heavy chain constant region, or a fragment thereof, and a hinge region, operably linked to a promoter which directs expression in mammary epithelial cells, wherein said hinge region has been altered from the hinge region normally associated with the heavy chain constant region.

2. The method of claim 1, wherein at least 70% of the antibodies present in the milk are in assembled form.

3. The method of claim 1, wherein said transgenic mammal further comprises a sequence encoding a light chain variable region, or antigen binding fragment thereof, and a light chain constant region or functional fragment thereof, operably linked to a promoter which directs expression in mammary epithelial cells.

4. The method of claim 1 further comprising the step of obtaining milk from said transgenic mammal, to thereby provide an antibody composition.

5. The method of claim 4 further comprising the step of purifying the exogenous antibody from the milk produced by said transgenic mammal.

6. The method of claim 1 wherein said promoter is a promoter selected from the group consisting of: casein promoter, lactalbumin promoter, beta lactoglobulin promoter and whey acid protein promoter.

7. The method of claim 1 wherein said transgenic mammal is a mammal selected from the group consisting of: cow, goat, mouse rat, sheep, pig and rabbit.

8. The method of claim 1 wherein the antibody is an antibody selected from the group consisting of: IgA, IgD, IgM, IgE or IgG.

9. The method of claim 1 wherein the antibody is an IgG antibody.

10. The method of claim 1 wherein the antibody is an IgG4 antibody.

11. The method of claim 10 wherein all or a portion of the hinge region of said antibody has been altered.

12. The method of claim 10, wherein all or a portion of the hinge region of the antibody has been replaced, e.g. replaced with a hinge region or portion thereof which differs from the hinge region normally associated with said heavy chain constant region.

13. The method of claim 10, wherein the amino acid sequence of the hinge region of the antibody differs from the amino acid sequence of the hinge region naturally associated with said heavy chain constant region by at least one amino acid residue.

14. The method of claim 1, wherein at least one of the nucleic acid residues of the nucleic acid sequence encoding the hinge region of the antibody differs from the naturally occurring nucleic acid sequence of the hinge region naturally associated with said heavy chain constant region.

15. The method of claim 12, wherein the hinge region of the antibody, or portion thereof, has been replaced with the hinge region, or portion thereof, of an antibody other than an IgG4 antibody.

16. The method of claim 12 wherein the hinge region, or portion thereof, of the antibody has been replaced with a hinge region, or portion thereof, derived from an antibody selected from a group consisting of: IgG1, IgG2 and IgG3.

17. The method of claim 12 wherein the hinge region of the antibody, or a portion thereof, has been replaced with a hinge region, or portion thereof, derived from an antibody selected from a group consisting of: IgA, IgD, IgM and IgE.

18. The method of claim 12 wherein one or more amino acids of the hinge region have been replaced with an amino acid corresponding to that position in an antibody other then an IgG4 antibody.

19. The method of claim 15 wherein the antibody other than an IgG4 antibody is an antibody selected from the group consisting of: IgA, IgD, IgM and IgE.

20. The method of claim 15 wherein the antibody other than an IgG4 antibody is an antibody selected from the group consisting of: IgG1, IgG2 and IgG3.

21. The method of claim 10, wherein a serine residue of the hinge region has been replaced with a proline residue.

22. The method of claim 10, wherein a serine residue at amino acid number 241 of the hinge region has been replaced with a proline residue.

23. The method of claim 10, wherein at least one amino acid in the hinge region other than a cysteine residue is replaced with a cysteine residue.

24. The method of claim 10 wherein at least 1 glycosylation site of the antibody is altered.

25. The method of claim 24, wherein at least one glycosylation site in the heavy chain or light chain is altered.

26. The method of claim 24, wherein at least one glycosylation site in the hinge region of the heavy chain is modified.

27. The method of claim 1 wherein the antibody is humanized.

28. The method of claim 1 wherein the antibody is chimeric.

29. The method of claim 1 wherein the antibody is a human antibody.

30. The method of claim 1 wherein the milk of the transgenic mammal is essentially free from a half molecule form of the exogenous antibody.

31. The method of claim 1 wherein the ratio of assembled exogenous antibody to half forms of the antibody present in the milk of a transgenic mammal are at least 2:1, 3:1, 4:1 or 5:1.

32. A method of producing a transgenic mammal whose somatic and germ cells comprise a modified antibody coding sequence wherein said modified antibody coding sequence encodes an antibody molecule or portion thereof expressible in milk, comprising a modified hinge region, said method comprising the steps of:
introducing into a mammal a construct comprising a sequence encoding an exogenous heavy chain variable region or antigen binding fragment thereof, at least one heavy chain constant region or a fragment thereof, and a hinge region, operably linked to a promoter which directs expression in mammary epithelial cells, wherein said hinge region has been altered from the hinge region normally associated with the heavy chain constant region.

33. The method of claim 33, wherein said hinge region has been altered such that at least 70% of the exogenous antibodies present in the milk of the transgenic mammal are in assembled form.

34. The method of claim 33, wherein said modified antibody coding sequence further comprises a sequence encoding a light chain variable region or antigen binding fragment thereof and a light chain constant region or functional fragment thereof, operably linked to a promoter which directs expression in mammary epithelial cells.

35. The method of claim 33 wherein the promoter is a promoter selected from the group consisting of: casein promoter, lactalbumin promoter, beta lactoglobulin promoter and whey acid protein promoter.

36. The method of claim 33 wherein the transgenic mammal is a mammal selected from the group consisting of: cow, goat, mouse rat, sheep, pig and rabbit.

37. The method of claim 33 wherein the antibody is an antibody selected from the group consisting of: IgA, IgD, IgM, IgE or IgG.

38. The method of claim 33 wherein the antibody is an IgG antibody.

39. The method of claim 33 wherein the antibody is an IgG4 antibody.

40. The method of claim 40 wherein all or a portion of the hinge region of the antibody has been altered.

41. The method of claim 40 wherein all or a portion of the hinge region of the antibody has been replaced, e.g. replaced with a hinge region or portion thereof which differs from the hinge region normally associated with said heavy chain variable region or said constant region.

42. The method of claim 40, wherein the amino acid sequence of the hinge region of the antibody differs from the amino acid sequence of the hinge region naturally associated with said heavy chain constant region by at least one amino acid residue.

43. The method of claim 33, wherein at least one of the nucleic acid residues of the nucleic acid sequence encoding the hinge region of the antibody differs from the nucleic acid sequence of the hinge region naturally associated with said heavy chain constant region.

44. The method of claim 44, wherein the hinge region of the antibody, or portion thereof, has been replaced with the hinge region, or portion thereof, of an antibody other than an IgG4 antibody.

45. The method of claim 42 wherein the hinge region, or portion thereof, of the antibody has been replaced with a hinge region, or portion thereof, derived from an antibody selected from a group consisting of: IgG1, IgG2 and IgG3.

46. The method of claim 42 wherein the hinge region of the antibody, or a portion thereof, has been replaced with a hinge region, or portion thereof, derived from an antibody selected from a group consisting of: IgA, IgD, IgM and IgE.

47. The method of claim 42 wherein one or more amino acids of the hinge region have been replaced with an amino acid corresponding to that position in an antibody other then an IgG4 antibody.

48. The method of claim 48 wherein the antibody other than an IgG4 antibody is an antibody selected from the group consisting of: IgA, IgD, IgM and IgE.

49. The method of claim 48 wherein the antibody other than an IgG4 antibody is an antibody selected from the group consisting of: IgG1, IgG2 and IgG3.

50. The method of claim 40, wherein a serine residue of the hinge region has been replaced with a proline residue.

51. The method of claim 40, wherein a serine residue at amino acid number 241 of the hinge region has been replaced with a proline residue.

52. The method of claim 40, wherein at least one amino acid in the hinge region other than a cysteine residue is replaced with a cysteine residue.

53. The method of claim 40 wherein at least one glycosylation site of the antibody is altered.

54. The method of claim 54 wherein at least one glycosylation site in the heavy chain or light chain is altered.

55. The method of claim 40, wherein at least one glycosylation site in the hinge region of the heavy chain is modified.

56. The method of claim 33 wherein the antibody is humanized.

57. The method of claim 33 wherein the antibody is a human antibody.

58. The method of claim 33 wherein the antibody is chimeric.

59. The method of claim 33, wherein said hinge region has been altered such that the milk of the transgenic mammal is essentially free from a half molecule form of the exogenous antibody.

60. The method of claim 33 wherein the ratio of assembled exogenous antibody to half forms of the antibody present in the milk of a transgenic mammal are at least 2:1, 3:1, 4:1 or 5:1.

61. The method of claim 60 wherein the antibody is an antibody selected from the group consisting of: IgA, IgD, IgM, IgE or IgG

62. A method of producing a transgenic mammal capable of expressing an assembled exogenous antibody or portion thereof in its milk, the method comprising:
introducing into a mammal a construct comprising a sequence encoding a light chain of exogenous antibody operably linked to a promoter which directs expression in mammary epithelial cells; and
introducing into the mammal a construct comprising a sequence encoding a mutagenized heavy chain of the exogenous antibody or a portion thereof operably linked to a promoter which directs expression in mammary epithelial cells, wherein the heavy chain or portion thereof comprises a hinge region which has been altered such that at least 70% of the exogenous antibodies present in the milk are in assembled form.

63. A method of producing a transgenic mammal capable of expressing an assembled exogenous antibody in its milk, the method comprising:
providing a cell from a transgenic mammal whose germ and somatic cells comprise a sequence encoding a light chain of an exogenous antibody operably linked to a promoter which directs expression in mammary epithelial cells; and
introducing into the cell a construct comprising a sequence encoding a mutagenized heavy chain of the exogenous antibody or a portion thereof operably linked to a promoter which directs expression in mammary epithelial cells, wherein the heavy chain, or portion thereof comprises a hinge region which has been altered such that at least 70% of the exogenous antibodies present in the milk are in assembled form.

64. A composition comprising a milk component and an antibody component, wherein said antibody component comprises an exogenous antibody, or fragment thereof, having a hinge region, wherein said hinge region has been altered from the hinge region normally associated with the antibody.

65. The composition of claim 63, wherein at least 70% of the exogenous antibodies present in said composition are in assembled form.

66. The composition of claim 63, wherein said hinge region has been altered such that at least 70% of the exogenous antibodies present in said composition in assembled form.

67. The composition of claim 63 wherein the antibody is an antibody selected from the group consisting of: IgA, IgD, IgM, IgE or IgG.

68. The composition of claim 63 wherein the antibody is an IgG antibody.

69. The composition of claim 67 wherein the antibody is an IgG4 antibody.

70. The composition of claim 63 wherein all or a portion of the hinge region of the antibody has been altered.

71. The composition of claim 63, wherein all or a portion of the hinge region of the antibody has been replaced, e.g. replaced with a hinge region or portion thereof which differs from the naturally occurring hinge region normally associated with the antibody.

72. The composition of claim 63, wherein the amino acid sequence of the hinge region of the antibody differs from the amino acid sequence of the hinge region of the naturally occurring antibody by at least one amino acid residue.

73. The composition of claim 63, wherein the hinge region of the antibody, or portion thereof, has been replaced with the hinge region, or portion thereof, of an antibody other than an IgG4 antibody.

74. The composition of claim 72 wherein the hinge region, or portion thereof, of the antibody has been replaced with a hinge region, or portion thereof, from an antibody selected from a group consisting of: IgG1, IgG2 and IgG3.

75. The composition of claim 72 wherein the hinge region of the antibody, or a portion thereof, has been replaced with a hinge region, or portion thereof, derived from an antibody selected from a group consisting of: IgA, IgD, IgM and IgE.

76. The composition of claim 63 wherein one or more amino acids of the hinge region have been replaced with an amino acid corresponding to that position in an antibody other then an IgG4 antibody.

77. The composition of claim 75 wherein the antibody other than an IgG4 antibody is an antibody selected from the group consisting of: IgA, IgD, IgM and IgE.

78. The composition of claim 75 wherein the antibody other than an IgG4 antibody is an antibody selected from the group consisting of: IgG1, IgG2 and IgG3.

79. The composition of claim 63, wherein a serine residue of the hinge region has been replaced with a proline residue.

80. The composition of claim 63, wherein a serine residue at amino acid number 241 of the hinge region has been replaced with a proline residue.

81. The composition of claim 63, wherein at least one amino acid in the hinge region other than a cysteine residue is replaced with a cysteine residue.

82. The composition of claim 63 wherein at least one glycosylation site of the antibody is altered.

83. The composition of claim 63, wherein at least one glycosylation site in the heavy chain or light chain of the antibody is altered.

84. The composition of claim 82, wherein at least one glycosylation site in the hinge region of the heavy chain of the antibody is modified.

85. The composition of claim 63 wherein the antibody is humanized.

86. The composition of claim 63 wherein the antibody is a human antibody.

87. The composition of claim 63, wherein said hinge region has been altered such that the composition is essentially free from a half molecule form of the exogenous antibody.

88. The composition of claim 63 wherein the ratio of assembled exogenous antibody to half forms of the antibody present in the composition is at least 2:1, 3:1, 4:1 or 5:1.

89. A nucleic acid comprising a sequence encoding a heavy chain variable region and a heavy chain constant region, operably linked to a promoter which directs expression in mammary epithelial cells, wherein the heavy chain or portion thereof comprises a hinge region which has been altered such that at least 70% of the exogenous antibodies present in milk are in assembled form.
